(19) United States
(12) Patent Application Publication
(10) Pub. No.: US 2023/0287396 A1
(43) Pub. Date: Sep. 14, 2023

(54) METHODS AND COMPOSITIONS OF NUCLEIC ACID ENRICHMENT

(71) Applicant: Honeycomb Biotechnologies, Inc., Waltham, MA (US)

(72) Inventors: Todd GIERAHN, Waltham, MA (US); Li CHEN, Waltham, MA (US)

(21) Appl. No.: 18/177,415

(22) Filed: Mar. 2, 2023

Related U.S. Application Data
(60) Provisional application No. 63/315,757, filed on Mar. 2, 2022.

(51) Int. Cl. C12N 15/10

(52) U.S. Cl. CPC C12N 15/1065

(57) ABSTRACT

This disclosure relates to compositions and methods for amplifying and identifying a target nucleic acid of interest from a population of non-target nucleic acids. In particular, this disclosure provides compositions and methods for identifying a target sequence from a single known adjacent sequence. This disclosure provides compositions and methods useful for identifying rare sequence variants, gene fusions, and for enriching and identifying nucleic acid whose target region of interest and its barcode for single-cell tracing are located on distant portions of a molecule.

### CROSS-REFERENCE

This application claims the benefit of U.S. Provisional Application No. 63/315,757, filed on Mar. 2, 2022, which is herein incorporated by reference in its entirety.

### BACKGROUND

A major barrier to treatment of many diseases is the inability to detect the disease at an early stage. Cancer, for example, results from changes in gene expression in individual cells that allow the cells to proliferate, invade other tissues, and hijack the body's resources. In early stages, however, the genetically altered cells represent a tiny fraction of the cells in a particular tissue or population. Consequently, the stage in which cells harboring deleterious mutations can be most easily eradicated is also the point at which the tumorigenic cells are most difficult to detect.

To facilitate early detection of diseased cells, many researchers have turned to single cell sequencing methodologies. The use of these methods, however, requires an ability to trace nucleic acid molecules back to the single cells from which they originate. This generally involves attaching unique barcode sequences to every nucleic acid molecule that is derived from the same cell. Unfortunately, this imposes difficult challenges for sequencing as it is difficult to maintain a barcode on one end of a transcript while identifying an unknown sequence on a distant end of the transcript.

### BRIEF SUMMARY

In view of the foregoing, there is a need for improved methods of detecting nucleic acid sequences of interest, such as rare sequence variants. The compositions and methods of the present disclosure address this need and provide additional advantages as well. In particular, the various aspects of the disclosure provide compositions (e.g., kits) and methods for amplifying and enriching target nucleic acids of interest, which can include nucleic acids harboring rare target sequence, rich in genetic diversity, or otherwise difficult to identify.

In one aspect, this disclosure provides a method of nucleic acid library preparation. The method includes: (a) amplifying a plurality of target nucleic acids and non-target nucleic acids with first primers to generate a plurality of first amplicons, wherein: (i) each of the plurality of target nucleic acids comprises a target sequence, and an adjacent region; (ii) the first primers each comprise one or more cleavage moieties between a 5′ and 3′ end; and (iii) the amplifying comprises a plurality of cycles of primer extension with the first primers to generate a plurality of double-stranded first amplicons for each of the plurality of target and non-target nucleic acids; (b) cleaving the plurality of first amplicons at the one or more cleavage moieties to produce cleaved amplicons with self-complementary 3′ overhangs; (c) circularizing the cleaved amplicons by ligating the ends at the self-complementary 3′ overhangs to generate circularized amplicons produced from the target nucleic acids and non-target nucleic acids; and (d) for each of a plurality of circularized amplicons comprising a target sequence, amplifying at least a portion of the circularized amplicon by extending one or more second primers wherein the one or more second primers preferentially hybridize to circularized amplicons produced from the target nucleic acids; thereby producing a nucleic acid library of second amplicons enriched for the target sequences and/or complements thereof.

In some embodiments, the plurality of cycles to generate the plurality of double-stranded first amplicons for each of the plurality of target and non-target nucleic acids comprises between 2 and 100 cycles. In some embodiments, the plurality of cycles comprises between 5 and 10 cycles. In some embodiments, the plurality of cycles comprises between 6 and 8 cycles. The plurality of cycles can be performed with primers that bind to primer binding sequences incorporated into the nucleic acid library during library preparation. In some embodiments, primer binding sites for the first primers comprise exogenous sequences that are the same for each of the plurality of target and non-target nucleic acids.

In some embodiments, the cleavage moiety comprises a uracil. In some embodiments, cleaving the plurality of first amplicons comprises excising the uracil.

In some embodiments, the first primers comprise a modification and are resistant to exonuclease digestion at one or more positions. In some embodiments, the modification comprises a phosphorothioate bond.

In some embodiments, each of the plurality of target nucleic acids encode at least a portion of a receptor selected from the group consisting of a T cell receptor, a B cell receptor, and a NK cell receptor. In some embodiments, the receptor is a T cell receptor or a B cell receptor, and wherein the target sequence comprises a variable region of said receptor. In some embodiments, the target nucleic acids comprise binding sites for the first primers that are located outside of the variable region.

In some embodiments, amplifying the circularized amplicons comprises binding the one or more second primers to the adjacent regions. In some embodiments, amplifying the circularized amplicons comprises binding the one or more second primers to the adjacent regions, wherein (i) the one or more second primers comprise a pair of second primers that hybridize to different complementary strands in the adjacent region of one or more of the circular amplicons, and (ii) the length in the 5′ to 3′ direction along one strand of the adjacent region defined by a binding site for one primer of the pair of second primers and a complement of a binding site for the other primer of the pair of second primers is less than 5 kb apart.

In some embodiments, the circularized amplicons are amplified by a single-stranded extension reaction. In some embodiments, amplifying the circularized amplicons comprises binding a single species of the one or more second primers to the adjacent regions and performing a single-stranded extension reaction with the single species of primers. In some embodiments, amplifying the circularized amplicons comprises between 2 and 22 cycles of amplification. In some embodiments, amplifying the circularized amplicons comprises between 18 and 24 cycles. In some embodiments, amplifying the circularized amplicons comprises 21 cycles.

In some embodiments, the method further comprises amplifying the nucleic acid library of second amplicons with pairs of third primers to produce a nucleic acid library of third amplicons. In some embodiments, amplifying the nucleic acid library of second amplicons comprises between 2 and 20 cycles of amplification. In some embodiments, amplifying the library of second amplicons comprises between 10 and 14 cycles of amplification. In some embodiments, amplifying the library of second amplicons comprises 13 cycles of amplification.

In some embodiments, each one of the pairs of third primers comprises a 5′ sequence and a 3′ sequence, wherein the 3′ sequence binds to a primer binding site nested with respect to the one or more second primers. In some embodiments, at least one primer of each of the pairs of third primers comprises a linker between the 5′ and 3′ sequences. In some embodiments, the linker is useful for increasing sequence diversity during sequencing. In some embodiments, the pairs of third primers comprise index sequences.

In some embodiments, the method of nucleic acid library preparation further comprises sequencing the library of second amplicons, or the library of third amplicons, to generate sequence reads; and identifying one or more of the target sequences. In some embodiments, the identifying comprises identifying a position of a sequence corresponding to the adjacent region. In some embodiments, the sequencing further comprises sequencing a barcode sequence, wherein the barcode sequence identifies a sample of origin of the associated target sequence. In some embodiments, the one or more of the target sequences comprises a gene fusion. In some embodiments, the gene fusion is identified by combining a first sequence read with a second sequence read to generate a chimeric sequence read wherein the first sequence read and the second sequence read mapped to two different regions of a reference genome. In some embodiments, the method further comprises measuring enrichment efficiency for the target sequence based on an analysis of sequences corresponding to the adjacent region.

In another aspect, this disclosure provides a method of gene profiling. The method comprises (a) constructing a library comprising a plurality of double stranded molecules of target cDNA and non-target cDNA with a poly-T primer, wherein each double stranded molecule of target cDNA comprises a target sequence and an adjacent region; (b) amplifying the plurality of double stranded molecules of target cDNA and non-target cDNA with first primers that comprise cleavage moieties to generate a plurality of first amplicons; (c) cleaving the plurality of first amplicons at the cleavage moieties to produce cleaved amplicons with self-complementary 3′ overhangs; (d) circularizing the cleaved amplicons by ligating the self-complementary 3′ overhangs to generate circularized amplicons produced from the target cDNA and non-target cDNA; and (e) for each of a plurality of the circularized amplicons comprising a target sequence, amplifying a portion of the circularized amplicons by extending one or more second primers, wherein the one or more second primers preferentially hybridize to circularized amplicons produced from the target cDNA; thereby producing a nucleic acid library of second amplicons enriched for the target sequences and/or complements thereof.

In some embodiments, the constructing comprises reverse transcribing mRNA from a cell with the poly-T primer and performing a template switching reaction to produce the plurality of double stranded molecules of target cDNA and non-target cDNA. In some embodiments, the constructing comprises reverse transcribing mRNA from a cell with the poly-T primer and then performing a second-strand synthesis reaction with a random primer.

In some embodiments of the method of gene profiling, the cell is selected from the group consisting of a T-cell, a B-cell, and a NK-cell. In some embodiments, the disclosure provides the method of gene profiling wherein (i) the plurality of double stranded molecules of target cDNA encode at least a portion of a receptor comprising a T-cell receptor or a B-cell receptor, and (ii) the target sequence of the target cDNA comprises a variable region of said receptor. In some embodiments, amplifying duplicates the entire variable region from each of a plurality of the double stranded molecules of cDNA.

In some embodiments, amplifying comprises a plurality of cycles of amplification with the first primers. In some embodiments, the plurality of cycles comprises between 2 and 100 cycles. In some embodiments, the plurality of cycles comprises between 5 and 10 cycles. In some embodiments, the plurality of cycles comprises between 6 and 8 cycles.

In some embodiments, the disclosure provides the method of gene profiling, wherein amplifying the circularized amplicons comprises between 2 and 22 cycles of amplification with the one or more second primers. In some embodiments, amplifying the circularized amplicons comprises between 20 and 22 cycles. In some embodiments, amplifying the circularized amplicons comprises between 21 cycles.

In some embodiments, the method of gene profiling further comprises amplifying the library of the second amplicons with one or more pairs of third primers to generate a library of third amplicons. In some embodiments, amplifying the library of second amplicons comprises between 2 and 15 cycles of amplification with the one or more pairs of third primers. In some embodiments, the constructing comprises reverse transcribing mRNA from a cell with the poly-T primer and performing random priming reaction to produce the plurality of double stranded molecules of target and non-target cDNA.

In some embodiments, the method of gene profiling further comprises sequencing the library of second amplicons, or the library of third amplicons, to generate sequence reads; and generating a gene profile of the cell with the sequence reads.

In another aspect, this disclosure provides a method of genotyping an immune cell. The method includes (a) amplifying one or more target nucleic acid molecules and non-target nucleic acid molecules from an immune cell with first primers to generate a library of first amplicons, wherein each of the one or more target nucleic acid molecules encodes a variable region of a receptor and an adjacent region of the receptor of the immune cell; (b) circularizing the plurality of first amplicons produced from the one or more target nucleic acid molecules and non-target nucleic acid molecules to generate circularized amplicons; and (c) amplifying a portion of the circularized amplicons by extending one or more primers across the variable regions to generate a nucleic acid library of second amplicons that is enriched for the variable regions. In some embodiments, the immune cell is a T-cell. In some embodiments, the immune cell is a B-cell.

In some embodiments of the method of genotyping the immune cell, the amplifying comprises a plurality of cycles. In some embodiments, the plurality of cycles comprises between 5 and 10 cycles. In some embodiments, the plurality of cycles comprises between 6 and 8 cycles. In some embodiments, each of the primers comprises a cleavage moiety. In some embodiments, circularizing the plurality of first amplicons comprises cleaving the plurality of first amplicons at the cleavage moiety to generate cleaved amplicons comprising self-complementary 3′ ends, and ligating the self-complementary 3′ ends.

In some embodiments of the method of genotyping the immune cell, the amplifying the circularized amplicons comprises binding the one or more second primers to the adjacent regions and extending the one or more primers across the variable regions. In some embodiments, the amplifying the circularized amplicons comprises between 2 and 22 cycles of amplification. In some embodiments, amplifying the circularized amplicons comprises between 20 and 22 cycles of amplification.

In some embodiments of the method of genotyping the immune cell, the method further comprises amplifying the library of second amplicons with one or more pairs of third primers to generate a library of third amplicons. In some embodiments, the amplifying the library of second amplicons comprises between 2 and 15 cycles of amplification.

In some embodiments of the method of genotyping the immune cell, the method further comprises amplifying the nucleic acid library of second amplicons, or the library of third amplicons, to generate a sequencing library; sequencing the sequencing library to generate a plurality of sequence reads; and genotyping the immune cell from the plurality of sequence reads.

In another aspect, this disclosure provides a kit for performing a methods described herein. The kit includes a DNA polymerase that is tolerant to uracil; one or more primer pairs; and a buffer.

In some embodiments, the kit further comprises at least one primer pair with sequences complementary to a portion of a T-cell receptor. In some embodiments, the kit further comprises at least one primer pair with sequences that are complementary to a portion of a house-keeping gene. In some embodiments, the kit further comprises an endonuclease and a ligase. In some embodiments, the kit further comprises indexing primers. In some embodiments, the kit further comprises beads for performing a library cleanup reaction. In some embodiments, the kit further comprises sequencing primers.

### BRIEF DESCRIPTION OF THE DRAWINGS

The novel features of the present disclosure are set forth with particularity in the appended claims. A better understanding of the features and advantages of the present disclosure can be obtained by reference to the following detailed description that sets forth illustrative embodiments, in which the principles of the disclosure are utilized, and the accompanying drawings of which:

FIGS. 1A-1B illustrate a method of preparing a nucleic acid library according to aspects of this disclosure.

FIG. 2 shows a genomic region of the TCR beta gene

FIG. 3 illustrates a method of preparing a nucleic acid library with nucleic acids that have difficult to access target regions.

FIG. 4 illustrates a method of nucleic acid library preparation for identifying a gene fusion with an unknown 5′ partner.

FIGS. 5A-5B illustrate certain limitations of single-cell RNA-seq that are overcome by circularization strategies disclosed herein. In particular, FIG. 5A illustrate several limitations associated with single cell RNA-seq with 3′ barcode tracing. FIG. 5B illustrates how circularization strategies of the present disclosure overcome those limitations.

FIGS. 6A-6B diagram three scenarios for target and non-target nucleic acid circularization and their corresponding impact on sequence analysis. In particular, FIGS. 6A-6B shows that the link between a single cell barcode and its target is preserved. Three scenarios illustrated herein include the intended single-copy circularization, the unintended tandem circularization with both copies being target molecules, and the unintended tandem circularization with one target and one non-target molecules. Unintended byproducts either rarely happen or can be distinguished.

FIG. 7 shows pre-circularization amplification data collected from reactions with various PCR cycle numbers and template input amounts.

FIG. 8 shows data from experiments optimizing USER/ligate input amounts.

FIG. 9 shows library yields from enrichment amplification reactions carried out using different PCR cycle numbers for the first and second enrichment step.

FIG. 10 shows data for identifying optimal enrichment and index cycle numbers.

FIG. 11 shows PCR data comparing concurrent vs consecutive PCR-2 and Index PCR reactions.

FIGS. 12A-12E show gel electrophoresis profiles of enrichment libraries prepared according to methods of the disclosure. FIG. 12A shows a gel comparing enrichment libraries enriched for GAPDH, TRAC, and TRBC, as compared to a whole transcriptome library. FIG. 12B shows the gel electrophoresis profile of TRAC. FIG. 12C shows the gel electrophoresis profile of GAPDH. FIG. 12D shows the gel electrophoresis profile of TRBC. FIG. 12E shows the gel electrophoresis profile of the whole transcriptome RNAseq library.

FIG. 13 shows data of sequencing quality of enrichment libraries.

FIG. 14 shows data generated by sequencing enrichment libraries.

FIG. 15 shows sequence data of libraries prepared with a single enrichment reaction compared to libraries prepared with two enrichment reactions.

FIG. 16 shows amplification data comparing various concentrations of primers for enrichment genes of interest.

FIG. 17 shows amplification data using different concentrations of a first set of primers (Primer-1 and Primer-2).

FIG. 18 shows target read ratios of sequenced libraries enriched for target sequences.

FIG. 19 shows a schematic workflow for preparing nucleic acid libraries according to aspects of this disclosure.

### DETAILED DESCRIPTION

This disclosure relates generally to compositions, kits, and methods useful for identifying nucleic acids of interest, including nucleic acids that-occur in trace amounts or are otherwise present at a low concentration within a population of non-target nucleic acids. In some aspects, this disclosure provides methods and compositions that are useful to enrich nucleic acids with minimal sequence information. In particular, certain aspects of this disclosure are useful to enrich target nucleic acids with only one known sequence. Accordingly, aspects of this disclosure provide strategies for identifying target nucleic acids, which are superior to traditional identification strategies, e.g., PCR. In certain embodiments, methods and compositions of this disclosure can be used to identify a target sequence without knowing the sequence of the target based on a single nucleic acid sequence adjacent to the target. In some embodiments, the target comprises a sequence of high sequence variability, e.g., a receptor of an immune cell. In some embodiments, methods and compositions described herein provide a more efficient manner of acquiring data from a variable region of T cell receptor (TCR) and/or a B cell receptor H/L chain (BCR) transcript. In some embodiments, the target comprises a portion of an unknown fusion gene. In some embodiments, methods described herein allow for the identification of fusion genes.

### Definitions

As used herein, “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, a “cell” refers to a biological cell. Some non-limiting examples include: a prokaryotic cell, eukaryotic cell, a bacterial cell, an archaea cell, a cell of a single-cell eukaryotic organism, a protozoa cell, a cell from a plant, an algal cell, a fungal cell, a fungal protoplast cell, an animal cell, and the like. Sometimes a cell is not originating from a natural organism, e.g., a cell can be a synthetically made, sometimes termed an artificial cell.

As used herein, the term “gene,” refers to a nucleic acid (e.g., DNA or RNA) and its corresponding nucleotide sequence that encodes a gene product, such as an RNA transcript or protein. The term as used herein with reference to genomic DNA includes intervening, non-coding regions as well as regulatory regions. The term encompasses the transcribed sequences, including 5 and 3 untranslated regions (5′-UTR and 3′-UTR), exons and introns.

As used herein, a “library” is a collection of nucleic acid molecules derived from one or more nucleic acid samples, in which fragments of nucleic acid may have been modified, such as by incorporating terminal adapter sequences comprising one or more primer binding sites and identifiable sequence tags. In some embodiments, a library comprises a collection of amplification products derived from amplifying one or more nucleic acid samples.

As used herein, a “linear extension reaction” refers to a reaction for the amplification of specific nucleic acid sequences by the extension of a single primer. A reaction involves one or more repetitions of the following steps: (i) denaturing the target nucleic acid, (ii) annealing a primer to a primer binding site, and (iii) extending the primer by a nucleic acid polymerase in the presence of nucleoside triphosphates. Usually, the reaction is cycled through different temperatures optimized for each step in a thermal cycler instrument. The reaction only produces as many single-stranded unidirectional product copies as the cycle number. One advantage of the reaction is that it possess high fidelity because each product is copied from the template and thus errors will not accumulate.

As used herein, the terms “nucleic acid”, “nucleotide”, “nucleotide sequence”, and “polynucleotide”, are used interchangeably. They refer to a polymeric form of nucleotides of any length, either deoxyribonucleotides or ribonucleotides, or analogs thereof. Nucleic acids may have any three dimensional structure, and may perform any function, known or unknown. The following are non-limiting examples of nucleic acids: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A nucleic acid may be further modified after polymerization, such as by conjugation with a labeling component

As used herein, the term “PCR” (polymerase chain reaction) refers to a reaction for the in vitro amplification of specific nucleic acid sequences by the simultaneous primer extension of complementary strands of nucleic acids. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps may depend on different factors. For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

As used herein, a “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

As used herein, a “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually, primers are extended by a DNA polymerase. Primers usually have a length in the range of between 3 to 36 nucleotides, for examples, from 10 to 24 nucleotides, or from 14 to 36 nucleotides. In certain aspects, primers are universal primers or non-universal primers. Pairs of primers can flank a sequence of interest or a set of sequences of interest. Primers and probes can be degenerate in sequence.

References to a percentage sequence identity between two nucleotide sequences means that, when aligned, that percentage of nucleotides are the same in comparing the two sequences. This alignment and the percent homology or sequence identity can be determined using software programs know in the art.

As used herein, “substantially pure” means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. In some embodiments, the compositions of the present disclosure are substantially pure.

In general, the term “target nucleic acid” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction.

As used herein, the term “cleavage moiety” refers to a part of a nucleic acid molecule that is acted upon by a protein or enzyme to result in the nucleic acid molecule being cleaved or nicked at the site of the cleavage moiety. In particular, the cleavage moiety can refer to a site of a nucleic acid molecule that is excised by the activities of a protein or enzyme. The cleavage moiety can be a nuclease-sensitive nucleotide, for example, a uracil.

Although various features of the disclosure may be described in the context of a single embodiment, the features can also be provided separately or in any suitable combination. Conversely, although the disclosure may be described herein in the context of separate embodiments for clarity, various aspects and embodiments can be implemented in a single embodiment.

Library Preparation

In some embodiments, nucleic acids are subjected to library preparation steps (e.g. circularization or amplification). In some embodiments, nucleic acids are subjected to certain library preparation steps without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of nucleic acids are available, such as by precipitation or non-specific binding to a substrate followed by washing the substrate to release bound polynucleotides. Where nucleic acids are isolated from a sample without a cellular extraction step, polynucleotides will largely be extracellular or “cell-free” nucleic acids, which may correspond to dead or damaged cells.

If a sample is treated to extract nucleic acids, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent, with or without the use of an automated nucleic acid extractor, e.g., the Model 341 DNA Extractor available from Applied Biosystems (Foster City, Calif.); (2) stationary phase adsorption methods; and (3) salt-induced nucleic acid precipitation methods, such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads.

In some embodiments, the above isolation methods may be preceded by an enzyme digestion step to help eliminate unwanted protein from the sample, e.g., digestion with proteinase K, or other like proteases. If desired, RNase inhibitors may be added to the lysis buffer. For certain cell or sample types, it may be desirable to add a proteindenaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

Where desired, nucleic acids from a sample may be fragmented prior to further processing. Fragmentation may be accomplished by any of a variety of methods, including chemical, enzymatic, and mechanical fragmentation. In some embodiments, the fragments have an average or median length from about 10 to about 1,000 nucleotides in length, such as between 10-800, 10-500, 50-500, 90-200, or 50-150 nucleotides. In some embodiments, the fragments have an average or median length of about or less than about 100, 200, 300, 500, 600, 800, 1000, or 1500 nucleotides. In some embodiments, the fragments range from about 90-200 nucleotides, and/or have an average length of about 150 nucleotides. In some embodiments, the fragmentation is accomplished mechanically comprising subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, restriction endonucleases, Cas endonucleases (e.g., Cas9), variants thereof, and combinations thereof. For example, digestion with DNase I can induce random double-stranded breaks in DNA in the absence of Mg++ and in the presence of Mn++. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. Fragmented polynucleotides may be subjected to a step of size selecting the fragments via standard methods such as column purification or isolation from an agarose gel.

In some embodiments, methods disclosed herein include constructing a library. The library can include a plurality of nucleic acids. In some embodiments, the library is enriched for target nucleic acids, each target nucleic acid comprising a target sequence. The library can further include nucleic acids comprising a unique molecular identifier (UMI), and/or a cell barcode. In some embodiments, each nucleic acid sequence is flanked by switching mechanism at 5′ end of RNA template (SMART) sequences at the 5′ end and/or 3′ end. The libraries can be constructed using any of a variety of single cell sequencing techniques, in some embodiments, an mRNA sequencing protocol, in some embodiments, SMART-Seq. Any of a variety of single cell sequencing protocols can be used, as described elsewhere herein, to construct the library. In some preferred embodiments, the protocol provides 3′ barcoded nucleic acids that are subjected to further steps.

In some embodiments, methods of constructing a library involve reverse transcribing RNA into cDNA. In some embodiments, methods include amplifying each nucleic acid in a library to create a whole transcriptome amplified (WTA) RNA by reverse transcription with primer, which can include an adapter. In some embodiments, the amplified RNA comprises the orientation: 5′-cell barcode/UMI-NNNNNNN-mRNA-3′, wherein N comprises a thymine or uracil. In some embodiments, PCR amplification is conducted with reverse transcribed products using primers that bind both sequence adapters and adding a library barcode and optionally additional sequence adapters. In some embodiments, the reverse transcribed products are amplified by PCR using primers comprise sequences that allow for subsequent circularization. In some embodiments, the reverse transcribed products are amplified by at least 1, 2, 3, 4, 5, 6, 7, 8, 9, 10, 11, 12, 13, 14, 15, 16, 17, 18, 19, 20 cycles of PCR. In some embodiments, the reverse transcribed products are reverse transcribed between 5-10 PCR cycles. For example, in some embodiments, the reverse transcribed products are PCR amplified by 8 cycles of PCR prior to circularizing.

In some embodiments, a library of nucleic acids comprising gene transcripts is provided, wherein the transcript comprises one or more barcodes at a 3′ end or a 5′ end. In some embodiments, a library of gene transcripts has portions of the transcript distant from a barcode, e.g., a 3′ cell barcode. The transcripts can be from, for example, a RNA-seq library. The generated library can contain desired transcripts, often enriched from low copy single cell sequencing, or from portions of a transcript that may be difficult to obtain in typical single-cell sequencing methods, while maintaining single cell identity. In some embodiments, the libraries contain variable regions of single cell matched T cell receptor a/P (TCR) or B cell receptor H/L chain (BCR) transcripts. In some embodiments, the library contains transcripts that are distant from the 3′ cell barcode, in some instances the library contains transcripts greater than about 1 kb away from the 3′ end of the transcript. In some embodiments, the enriched libraries are prepared by enrichment of transcripts containing gene mutations located anywhere in the genome.

Target Nucleic Acids

In some embodiments, compositions and methods disclosed herein are useful in identifying nucleic acids, including double-stranded DNA, single-stranded DNA, single-stranded DNA hairpins, DNA/RNA hybrids, RNAs with a recognition site for binding of the polymerizing agent, and RNA hairpins. Further, a target nucleic acid may be a specific portion of a genome of a cell, such as an intron, regulatory region, allele, variant or mutation; the whole genome; or any portion therebetween. In some embodiments, the target nucleic acid may be mRNA, tRNA, rRNA, ribozymes, antisense RNA or RNAi. The target nucleic acid may be of any length, such as at least 10 bases, at least 25 bases, at least 50 bases, at least 100 bases, at least 500 bases, at least 1000 bases, or at least 2500 bases. In some embodiments, the target nucleic acid comprises a deletion. Embodiments disclosed herein are particularly useful in high throughput sequencing of single molecule nucleic acids in which a plurality of target polynucleotides are attached to a solid support in a spatial arrangement such that each polynucleotide is individually optically resolvable.

A target nucleic acid may comprise target sequence, such as a gene of interest or a portion thereof. A target sequence can refer to any polynucleotide, such as DNA or RNA polynucleotides. In some embodiments, a target sequence is derived from the nucleus or cytoplasm of a cell, and may include nucleic acids in or from mitochondrial, organelles, vesicles, liposomes or particles present within the cell and subjected to a single cell sequencing method, retaining identification of the source cell or subcellular organelle. A target sequence may comprise, for example, a mutation, deletion, insertion, translocation, single nucleotide polymorphism (SNP), splice variant or any combination thereof associated with a particular attribute in a gene of interest. In some embodiments, the target sequence may encode a cancer gene. In some embodiments, the target sequence is a mutated cancer gene, such as a somatic mutation. Conversely, a non-target nucleic acid may refer to any nucleic acid that is not of interest. For example, a non-target nucleic acid can refer to any polynucleotide, such as DNA or RNA, that does not comprise, for example, a mutation, deletion, insertion, translocation, single nucleotide polymorphism (SNP), splice variant or any combination thereof associated with a particular attribute in a gene of interest. As a further example, target sequences may be derived from one or more genes of interest or portions thereof, and polynucleotides not derived from the one or more genes of interest or portions thereof (e.g., other genes, or intergenic regions) are non-target sequences. In some embodiments, a target nucleic acid is enriched for relative to the non-target nucleic acid.

In some embodiments, a target sequence comprises a mutation. In some embodiments, the mutation is located anywhere in a gene, or a regulator of the gene (e.g., an enhancer or gene promoter). In some embodiments, the desired target sequence can be greater than about 1 kb away from a cell barcode of the nucleic acid of the libraries as described here. The target sequence may comprise a SNP.

In some embodiments, a library of target and non-target nucleic acids can include a target sequence. The target sequence can comprise a portion of the target nucleic acid. The target sequence can be any length. For example, the target sequence can be 10, 20, 30, 40, 50, 100, 200, 300, 400, 500, 1000, or more nucleotides in length. In some embodiments, the target sequence encodes a gene of a T cell or a B cell or an NK cell. In some embodiments, the target sequence comprises a T cell receptor, a B cell receptor, an NK cell receptor, or a CAR-T cell. In some embodiments, the target sequence comprises a variable region of a T cell receptor or a B cell receptor.

Pre-Circularization Amplification

In some embodiments, methods of the disclosure involve amplifying nucleic acids prior to circularization. In some embodiments, methods of the disclosure involve amplifying target and non-target nucleic acids prior to circularization. In particular, it is an insight of the disclosure that the amplification of non-target nucleic acids with target nucleic acids produces higher quality sequencing libraries of nucleic acids enriched for the target nucleic acids. Without limiting the scope of the disclosure, one hypothesis for the higher quality libraries produced by amplifying non-target nucleic acids in combination with the target nucleic acids is that the presence of non-target nucleic acids in a circularization reaction substantially reduces opportunities for two different molecules of target nucleic acids to join together, which reduces sequence read fidelity. By amplifying non-target nucleic acids with target nucleic acids opportunities for two molecules of target nucleic acids to join and circularize together are reduced. As discussed below, circularization of a non-target nucleic acid joined with another non-target nucleic acid, or circularization of a non-target nucleic acid joined with a target nucleic acid can be filtered, and therefore the presence of non-target nucleic acids does not negatively impact sequence read fidelity.

In some embodiments, the nucleic acids are amplified with primers that mediate downstream circularization. In some embodiments, the primers comprise complementary sequences on 5′ ends, and one or more cleavage moieties between the 5′ end and the 3′ end. In some embodiments, the one or more cleave moieties comprise one or more deoxy-uracil residues. In such embodiments, circularizing can comprise reacting the amplicons with a cleavage reagent (e.g., a uracil-specific excision reagent enzyme), thereby cleaving the amplicon at the deoxy-uracil residues resulting in sticky ends that can be ligated to accomplish circularization.

In some embodiments, pre-circularized amplification is achieved using a polymerase chain reaction (PCR). PCR encompasses derivative forms of the reaction, including but not limited to, RT-PCR, real-time PCR, nested PCR, quantitative PCR, multiplexed PCR, and the like. PCR makes use of a polymerizing agent (DNA polymerase) to extend primers bound to a nucleic acid with free dNTPs. A variety of such polymerases are available, non-limiting examples of which include exonuclease minus DNA Polymerase I large (Klenow) Fragment, Phi29 DNA polymerase, Taq DNA Polymerase and the like. The amplification primers may be of any suitable length, such as about or at least about 5, 10, 15, 20, 25, 30, 35, 40, 45, 50, 55, 60, 65, 70, 75, 80, 90, 100, or more nucleotides, any portion or all of which may be complementary to the corresponding target sequence to which the primer hybridizes (e.g. about, or at least about 5, 10, 15, 20, 25, 30, 35, 40, 45, 50, or more nucleotides). In some embodiments, multiple target-specific primers for a plurality of targets are used in the same reaction. For example, target-specific primers for about or at least about 10, 50, 100, 150, 200, 250, 300, 400, 500, 1000, 2500, 5000, 10000, 15000, or more different target sequences may be used in a single amplification reaction in order to amplify a corresponding number of target sequences (if present) in parallel. Multiple target sequences may correspond to different portions of the same gene, different genes, or non-gene sequences. Where multiple primers target multiple target sequences in a single gene, primers may be spaced along the gene sequence (e.g. spaced apart by about or at least about 50 nucleotides, every 50-150 nucleotides, or every 50-100 nucleotides) in order to cover all or a specified portion of a target gene.

The PCR reaction may involve any number of PCR cycles. For example, in some embodiments, the plurality of cycles comprises between 2 and 100 cycles. For example, in some embodiments, the plurality of cycles comprises 2, 3, 4, 5, 6, 7, 8, 9, 10, 11, 12, 13, 14, 15, 16, 17, 18, 19, 20, 25, 30, 35, 40, 45, 50, or 100 cycles. In some embodiments, the plurality of cycles comprises between 5 and 10 cycles. In some embodiments, the plurality of cycles comprises 8 cycles.

In some embodiments, the primers for amplifying in a first PCR amplification comprise USER sequences, and further comprising treating the first PCR product with USER enzyme and joining resulting self-complementary ends (e.g., by intra-molecular ligation), thereby generating a circularized product. The primers may be referred to herein as “Primer-1”, “Primer-2”, “Primer-3”, “Primer-4”, “Primer-5”, and “Primer-6”. For example, first primers may be referred to as “Primer-1” and “Primer-2”. One or more second primers, for example, as used in an Enrichment-1 reaction, may be referred to as “Primer-3” and “Primer-4”. One or more third primers, for example, as used in an Enrichment-2 reaction, may be referred to as “Primer-5” and “Primer-6”.

Illustrative steps include cleaving the dU residue by addition of a uracil-specific excision reagent (“USER®”) enzyme/T4 ligase to generate long complementary sticky ends to mediate efficient circularization and ligation, which can place a barcode and the 5′ edge of the transcript sequence set in the primer extension in close proximity, thereby bringing the cell barcode within 100 bases of any desired sequence in the transcript.

Following treating with USER enzyme, the step of amplifying the circularized product in a second polymerase chain reaction with one or more primers, wherein the one or primers comprise a library barcode and/or additional sequencing adapters can be conducted.

Circularization

According to some embodiments, polynucleotides among the plurality of polynucleotides from a sample are circularized. Circularization can include joining the 5′ end of a nucleic acid to the 3′ end of the same nucleic acid. In some embodiments, the 5′ end of a polynucleotide is joined to the 3′ end of the same polynucleotide (also referred to as “self-joining”). In some embodiment, conditions of the circularization reaction are selected to favor self-joining of target nucleic acids. In some embodiments, conditions of circularization can be selected to favor self-joining involve diluting nucleic acids present in a reaction mixture by adding buffer. By diluting nucleic acids, the probability that two nucleic acid molecules will be in the same vicinity during circularization is reduced. In some embodiments, the nucleic acids are diluted 5, 10, 15, 20, 25, 50, or 75 percent. In some embodiments, conditions of circularization are selected to favor self-joining of nucleic acids within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, temperature of a joining reaction, the concentration of various reagents, and the concentration of polynucleotides to be joined. In some embodiments, a circularization reactions preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

In some embodiments, circularizing comprises reacting the amplicons with a uracil-specific excision reagent enzyme, thereby cleaving the amplicon at the deoxy-uracil residues resulting in sticky ends that mediate circularization. In some embodiments, the amplicons are contacted with a uracil-specific excision reaction to generate sticky ends which are subsequently ligated to generate circularized amplicons. For example, circularizing the amplicons can involve treating the amplicons with an enzyme mix sold under the trade name USER Enzyme by New England Biolabs. The enzyme mix may comprise a mixture of uracil DNA glycosidase (UDG) and DNA glycosylase-lyase endonuclease VIII. In some embodiments, the amplicons are treated with uracil DNA glycosidase (UDG), which excises uracil bases selectively while leaving the phosphodiester backbone intact. Next, a T4 endonuclease V may be used to break at the DNA phosphodiester backbone at the 3′ side of an abasic site. Accordingly, in some embodiments, Therefore, sequential activity of UDG and T4 endonuclease V on PCR products amplified with primers containing at least one uracil residue is used to generate complementary overhangs that can be used efficiently for circularization.

A variety of methods for circularizing nucleic acids are available. In some embodiments, circularization comprises treating the amplicons with an endonuclease that cleaves a target nucleic acid at a specific site leaving blunt ends, or complementary overhangs. In some embodiments, the endonuclease is a Cas endonuclease. For example, in some embodiments, the endonuclease is a Cas9 endonuclease. In some embodiments, the circularization comprises treating the amplicons with a restriction enzyme. A restriction enzyme, restriction endonuclease, or restrictase is an enzyme that cleaves DNA into fragments at or near specific recognition sites within molecules known as restriction sites. In some embodiments, circularization is performed by ligating blunt ended fragments. In some embodiments, circularization is performed by ligating complementary overhangs. Ligating the ends of fragments can be performed with a ligase (e.g. an RNA or DNA ligase). A variety of ligases are available, including, but not limited to, Circligase™ (Epicentre; Madison, Wis.), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. Where self-joining is desired, the concentration of polynucleotides and enzyme can be adjusted to facilitate the formation of intramolecular circles rather than intermolecular structures. Reaction temperatures and times can be adjusted as well. In some embodiments, 60 degrees Celsius is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I). Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

Circularization may be followed directly by sequencing the circularized polynucleotides. Alternatively, sequencing may be preceded by one or more amplification reactions. In general, “amplification” refers to a process by which one or more copies are made of a target polynucleotide or a portion thereof. A variety of methods of amplifying polynucleotides (e.g. DNA and/or RNA) are available.

Enrichment (Enrichment-1 and/or Enrichment-2 Reactions)

After circularization, reaction products may be enriched for portions of the circularized amplicons comprising target sequences. The target sequences can be enriched for by binding and extending primer pairs, e.g., one or more second primers (e.g., Primer-3, Primer-4) that hybridize to a known sequence (adjacent region) adjacent to the target sequences. As described herein, enrichment reactions are sometimes referred to as Enrichment-1 and Enrichment-2 reactions.

In some embodiments, circularized amplicons can be purified prior to enrichment or sequencing to increase the relative concentration or purity of circularized amplicons available for participating in subsequence steps (e.g. by isolation of circular amplicons or removal of one or more other molecules in the reaction). For example, a circularization reaction or components thereof may be treated to remove single-stranded (non-circularized) polynucleotides, such as by treatment with an exonuclease. As a further example, a circularization reaction or portion thereof may be subjected to size exclusion chromatography, whereby small reagents are retained and discarded (e.g. unreacted adapters), or circularization products are retained and released in a separate volume. A variety of kits for cleaning up ligation reactions are available, such as kits provided by Zymo oligo purification kits made by Zymo Research. In some embodiments, purification comprises treatment to remove or degrade ligase used in the circularization reaction, and/or to purify circularized polynucleotides away from such ligase. In some embodiments, Solid Phase Reversible Immobilization (SPRI) beads are used for library clean up. SPRI beads use paramagnetic beads to selectively bind to nucleic acids by type and size, and can be used for high-performance isolation, purification, and cleanup protocols. In particular, SPRI beads can be added to a reaction mixture of nucleic acids, incubated for a period of time to allow for the beads to bind with the nucleic acids, washed, and removed using a magnet. In some embodiments, treatment to degrade ligase comprises treatment with a protease, such as proteinase K. Proteinase K treatment may follow manufacturer protocols, or standard protocols (e.g. as provided in Sambrook and Green, Molecular Cloning: A Laboratory Manual, 4th Edition (2012)). Protease treatment may also be followed by extraction and precipitation. In one example, circularized polynucleotides are purified by proteinase K (Qiagen) treatment in the presence of 0.1% SDS and 20 mM EDTA, extracted with 1:1 phenol/chloroform and chloroform, and precipitated with ethanol or isopropanol. In some embodiments, precipitation is in ethanol.

Enrichment generally involves amplification with one or more primers that flank target sequences. Amplification may be linear, exponential, or involve both linear and exponential phases in a multi-phase amplification process. Amplification methods may involve changes in temperature, such as a heat denaturation step, or may be isothermal processes that do not require heat denaturation. The polymerase chain reaction (PCR) uses multiple cycles of denaturation, annealing of primer pairs to opposite strands, and primer extension to exponentially increase copy numbers of the target sequence. Denaturation of annealed nucleic acid strands may be achieved by the application of heat, increasing local metal ion concentrations, and application of an electromagnetic field in combination with primers bound to a magnetically-responsive material. In some embodiments, a single enrichment reaction is performed. In some embodiments, a first and a second enrichment reaction is performed. In some embodiments, 1, 2, 3, 4, 5, or more enrichment reactions are performed.

Target sequences of interest can be enriched for by extending one or more primers that bind to regions flanking the target sequences. In some embodiments, the one or more primers bind to adjacent regions. In some embodiments, the primers bind to adjacent regions and are subsequently extended by PCR. In some embodiments, enriching for a target of interest involves a plurality of PCR cycles. For example, in some embodiments, the plurality of cycles comprises between 2 and 30 cycles of primer extension. In some embodiments, the plurality of cycles comprises between 2 and 20 cycles. In some embodiments, the plurality of cycles comprises between 12 and 20 cycles. In some embodiments, the plurality of cycles comprises 2, 3, 4, 5, 6, 7, 8, 9, 10, 11, 12, 13, 14, 15, 16, 17, 18, 19, or 20 cycles of primer extension. In some embodiments, the target sequence is enriched at least 2 fold relative to its content before the enrichment. In some embodiments, the target sequence is enriched at least 10, 10², 10³, 10⁴, or 10⁵ relative to its content before the enrichment. In some embodiments, the method further comprises sequencing the library of second amplicons, or the library of third amplicons, to generate sequence reads; and generating a gene profile of the cell with the sequence reads.

Sequencing

According to some embodiments, circularized polynucleotides (or amplification products thereof, which may have optionally been enriched) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product.

In some embodiments, sequencing involves detecting the incorporation of differently labeled nucleotides, which is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β,γ, or other terminal phosphate group on a nucleoside polyphosphate, the label is not incorporated into the nascent strand, and instead, natural DNA is produced.

According to some embodiments, a sequence difference between sequencing reads and a reference sequence are called as a sequence variant (e.g. existing in the sample prior to amplification or sequencing, and not a result of either of these processes) if it occurs in at least two different polynucleotides (e.g. two different circular polynucleotides, which can be distinguished as a result of having different junctions). Because sequence variants that are the result of amplification or sequencing errors are unlikely to be duplicated exactly (e.g. position and type) on two different polynucleotides comprising the same target sequence, adding this validation parameter greatly reduces the background of erroneous sequence variants, with a concurrent increase in the sensitivity and accuracy of detecting actual sequence variation in a sample. In some embodiments, a sequence variant having a frequency of about or less than about 5%, 4%, 3%, 2%, 1.5%, 1%, 0.75%, 0.5%, 0.25%, 0.1%, or lower is sufficiently above background to permit an accurate call. In some embodiments, the sequence variant occurs with a frequency of about or less than about 0.1%. In some embodiments, the frequency of a sequence variant is sufficiently above background when such frequency is statistically significantly above the background error rate (e.g. with a p-value of about or less than about 0.05, 0.01, 0.001, 0.0001, or lower). In some embodiments, the frequency of a sequence variant is sufficiently above background when such frequency is about or at least about 2-fold, 3-fold, 4-fold, 5-fold, 6-fold, 7-fold, 8-fold, 9-fold, 10-fold, 25-fold, 50-fold, 100-fold, or more above the background error rate (e.g. at least 5-fold higher). In some embodiments, the background error rate in accurately determining the sequence at a given position is about or less than about 1%, 0.5%, 0.1%, 0.05%, 0.01%, 0.005%, 0.001%, 0.0005%, or lower. In some embodiments, the error rate is lower than 0.001%.

In some embodiments, identifying a sequence variant (also referred to as “calling” or “making a call”) comprises optimally aligning one or more sequencing reads with a reference sequence to identify differences between the two, as well as to identify junctions. In general, alignment involves placing one sequence along another sequence, iteratively introducing gaps along each sequence, scoring how well the two sequences match, and preferably repeating for various positions along the reference. The best-scoring match is deemed to be the alignment and represents an inference about the degree of relationship between the sequences. In some embodiments, a reference sequence to which sequencing reads are compared is a reference genome, such as the genome of a member of the same species as the subject. A reference genome may be complete or incomplete. In some embodiments, a reference genome consists only of regions containing target polynucleotides, such as from a reference genome or from a consensus generated from sequencing reads under analysis. In some embodiments, a reference sequence comprises or consists of sequences of polynucleotides of one or more organisms, such as sequences from one or more bacteria, archaea, viruses, protists, fungi, or other organism. In some embodiments, the reference sequence consists of only a portion of a reference genome, such as regions corresponding to one or more target sequences under analysis (e.g. one or more genes, or portions thereof). For example, for detection of a pathogen (such as in the case of contamination detection), the reference genome is the entire genome of the pathogen (e.g. HIV, HPV, or a harmful bacterial strain, e.g. E. coli), or a portion thereof useful in identification, such as of a particular strain or serotype. In some embodiments, sequencing reads are aligned to multiple different reference sequences, such as to screen for multiple different organisms or strains.

In a typical alignment, a base in a sequencing read alongside a non-matching base in the reference indicates that a substitution mutation has occurred at that point. Similarly, where one sequence includes a gap alongside a base in the other sequence, an insertion or deletion mutation (an “indel”) is inferred to have occurred. When it is desired to specify that one sequence is being aligned to one other, the alignment is sometimes called a pairwise alignment. When individual bases are aligned, a match or mismatch contributes to the alignment score by a substitution probability, which could be, for example, 1 for a match and 0.33 for a mismatch. An indel deducts from an alignment score by a gap penalty, which could be, for example, −1. Gap penalties and substitution probabilities can be based on empirical knowledge or a priori assumptions about how sequences mutate. Their values affect the resulting alignment. A non-limiting example of an algorithm for performing alignments includes the Smith-Waterman (SW) algorithm.

Typically, the sequencing data is acquired from large scale, parallel sequencing reactions. Many of the next generation high-throughput sequencing systems export data as FASTQ files, although other formats may be used. In some embodiments, sequences are analyzed to identify repeat unit length (e.g. the monomer length), the junction formed by circularization, and any true variation with respect to a reference sequence, typically through sequence alignment. Identifying the repeat unit length can include computing the regions of the repeated units, finding the reference loci of the sequences (e.g. when one or more sequences are particularly targeted for amplification, enrichment, and/or sequencing), the boundaries of each repeated region, and/or the number of repeats within each sequencing run. Sequence analysis can include analyzing sequence data to identify a gene fusion, which can involve mapping sequence reads of contiguous nucleotides to portions of a chromosome of a reference genome that separated by a number of nucleotides, or mapping sequence reads of contiguous bases to different chromosomes of a reference genome. Sequence analysis can include identifying a variant. The sequence variant in the nucleic acid sample can be any of a variety of sequence variants. Multiple non-limiting examples of sequence variants are described herein, such as with respect to any of the various aspects of the disclosure. In some embodiments the sequence variant is a single nucleotide polymorphism (SNP). In some embodiments, the sequence variant occurs with a low frequency in the population (also referred to as a “rare” sequence variant). For example, the sequence variant may occur with a frequency of about or less than about 5%, 4%, 3%, 2%, 1.5%, 1%, 0.75%, 0.5%, 0.25%, 0.1%, or lower. In some embodiments, the sequence variant occurs with a frequency of about or less than about 0.1%.

In some embodiments, sequencing is performed using unique molecular identifiers (UMI). The term “unique molecular identifiers” (UMI) as used herein refers to a sequencing linker or a subtype of nucleic acid barcode used in a method that uses molecular tags to detect, distinguish, and/or quantify unique amplified products. In certain embodiments, an UMI with a random sequence of between 4 and 20 base pairs is added to a template, which is amplified and sequenced.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI. In some embodiments, a UMI is the combination of an exogenous index sequence and a portion of a sequence derived from a sample nucleic acid to which it was joined. Thus, a particular index sequence may be used more than once, but association of the redundant index with a different target or junction sequence renders the combination unique for use as a UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt.

In some embodiments, this disclosure offers useful strategies to overcome problems in genomic analysis that arise from an inability to distinguish identical or nearly identical template sequences. Because read lengths are generally short, determining the physical connection of distinguishable elements separated by long identical stretches can be difficult to impossible by prior methods, and limits our ability to phase single nucleotide variants (SNVs), and assemble through repetitive genomic regions. To address these issues, this disclosure offers embodiments utilizing “position counting” as a method of transcript counting and origin tracing.

In some embodiments, this disclosure involves “position counting” in which different transcript molecules are distinguished by the positions of their 5′ and/or 3′ ends. The ends can be generated either through fragmentation, natural degradation, biological events such as alternative splicing, or through random priming in a first-strand cDNA synthesis reaction. This method is useful for methods of this disclosure because, by choosing the middle of a gene as primer-binding region, the end positions are preserved throughout the enrichment process. As such, the ends generated by fragmenting a sample of nucleic acids are carried through subsequent amplification steps during library preparation and thus can be used to trace sequence reads to their molecule of origin. In some embodiments, methods make use of a fragment's 5′ end for position counting. In some embodiments, methods make use of a fragment's 3′ end. In some embodiments, methods make use of a fragment's 5′ and 3′ end for position counting.

### Kits

In one aspect, provided herein are kits that comprise one or more compositions or agents described herein. For example, in one aspect a kit described herein comprises compositions or agents for preparing a nucleic acid library according to any one of the methods described herein. In some embodiments, the kit can include compositions and agents for preparing a nucleic acid library from nucleic acids released from a cell. In some embodiments, the kit can include reagents for pre-circularization amplification. Accordingly, in some embodiments, the kit can include primers. In some embodiments, the primers may comprise one or more uracil residues. In some embodiments, the kit can comprise a DNA polymerase that is tolerant to a uracil. In some embodiments, the kit can include compositions and agents for circularizing a nucleic acid. Accordingly, in some embodiments, the kit can include a uracil excision reagent, e.g., an endonuclease. In some embodiments, the kit can include a ligase. In some embodiments, the kit can include reagents for performing one or more enrichment reactions. In some embodiments, the kit can include reagents for purifying nucleic acid or performing a library cleanup step. In some embodiments, said kit comprises cells for use in assays described herein, including, but not limited to, immune cells (e.g., T cells). In some embodiments, said kit comprises reagents to determine a read out from an in vitro assay described herein (e.g., in vitro cytolytic activity of a plurality of T cells). In some embodiments, said kit comprises instructions for preparing a nucleic acid library as described herein an assay described herein.

Exemplary components of kits provided herein are shown in the Tables 1-5 below.

TABLE 1
Components of an exemplary kit for preparing nucleic acid library according to methods
disclosed herein. The kit described below can be used for preparing 24 reactions.

| | | usage per | | |
|---|---|---|---|---|
| Reagent | Qty | reaction | | Description |
| 10 mM Tris | 1.8 mL × 2 | ~80 | uL | Use throughout process |
| Pre-circ Amplification | 360 uL × 1 | 12.5 | uL | enzyme for pre-circularization PCR |
| Enzyme | | | | |
| Pre-circ Primer Mix | 150 uL × 1 | 5 | uL | Primer mix for pre-circularization PCR |
| 10× Circularization | 60 uL × 1 | 2 | uL | Buffer for circularization reaction |
| Buffer | | | | |
| Circularization Enzyme | 60 uL × 1 | 2 | uL | Enzyme mix for circularization reaction |
| PCR Enzyme | 1.8 mL × 1 | 50 | uL | Use for both Enrichment-1 and Enrichment- |
| | | | | 2/indexing PCR |

| | | | |
|---|---|---|---|
| Circ Index 1 seqP | 100 uL × 1 | ~5-10 uL per | Custom Index1 sequencing primer for |
| | | sequencing | circularization kit |
| Circ Read 2 seqP | 100 uL × 1 | ~5-10 uL per | Custom Read2 sequencing primer for |
| | | sequencing | circularization kit |

TABLE 2
Components of an exemplary kit for
performing a library cleanup step.

| | | usage per | |
|---|---|---|---|
| Reagent | Qty | reaction | description |
| SPRI Beads | 5 mL × 1 | 40 uL | Use for library clean up |

TABLE 3
Components of an exemplary kit for
performing an indexing reaction.

| | | usage per | |
|---|---|---|---|
| Reagent | Qty | reaction | description |
| Universal UDI Index | 25 uL × 96- | 15 uL × 1 | Use for index |
| Plate for Illumina | well plate | well | PCR reaction |

TABLE 4
Components of an exemplary kit for
hTCR-specific target enrichment.

| | | usage per | |
|---|---|---|---|
| Reagent | Qty | reaction | description |
| hTCR Enrichment-1 | 150 uL × 1 | 5 uL | Use for human TCR |
| Primer Mix | | | Enrichment-1 PCR |
| hTCR Enrichment-2 | 450 uL × 1 | 5 uL | Use for human TCR |
| Primer Mix for | | | Enrichment-2/indexing |
| Illumina | | | PCR |

TABLE 5
Components of an exemplary kit.

| | | usage per | |
|---|---|---|---|
| Reagent | Qty | reaction | description |
| Human housekeeping control | 150 uL × 1 | 5 uL | Use for control |
| Enrichment-1 Primer Mix | | | Enrichment-1 |
| | | | PCR |
| Human housekeeping control | 450 uL × 1 | 5 uL | Use for control |
| Enrichment-2 Primer Mix for | | | Enrichment-2/ |
| Illumina | | | indexing PCR |

### Exemplary Embodiments

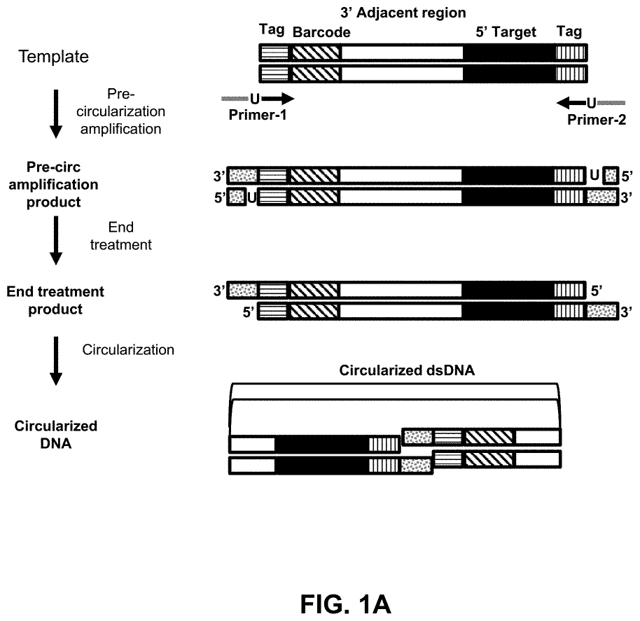

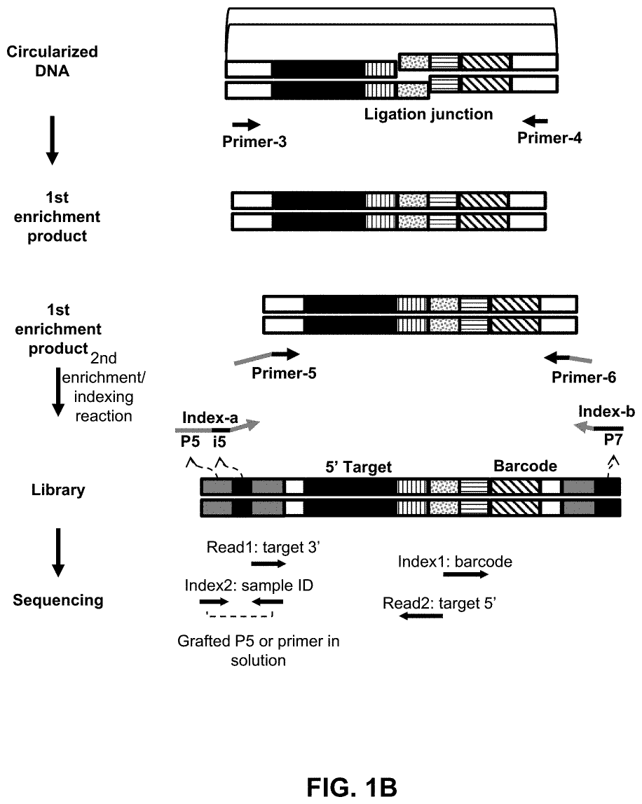

FIGS. 1A-1B illustrate a method of preparing a nucleic acid library according to certain aspects of this disclosure. The method can be used to enrich for a target of interest. In some embodiments, the method can be used to enrich for a target of interest even without knowing the exact sequence of the target. In some embodiments, the method can be used to enrich for a target of interest wherein the target of interest comprises sequences that are difficult to access or bind with primers. In some embodiments, the enriched target can be identified by sequencing. In some embodiments, the method allows for enrichment of a target sequence in instances where only one side of the target sequence (i.e., the adjacent region) is known or otherwise available for specific selection. For example, in some embodiments, the method allows for the enrichment and identification of a target sequence using an adjacent region comprising a known sequence. Furthermore, the method can allow for the target sequence to be associated with a sequence identifier (e.g., a UMI or barcode) present on the opposite end of the adjacent region. Accordingly, as one skilled in the art will readily appreciate, the illustrated method is useful for any number of applications in which the enrichment of a target sequence is advantageous.

In general, the method involves the following steps: pre-circularization amplification, circularization, enrichment (e.g., with one or two enrichment reactions), and sequencing. In some embodiments, the method begins with amplifying target and non-target (not shown) nucleic acids with a first set of primers (Primer-1 and Primer-2) which comprise cleavage moieties. In some embodiments, the cleavage moieties comprise one or more nuclease sensitive nucleotides. In some embodiments, the cleavage moieties comprise deoxy-uracil residues.

The target and non-target nucleic acids can be any type of nucleic acid. For example, without limiting the scope of the present disclosure, the nucleic acid can be DNA, for example, the nucleic acid can be complementary DNA (cDNA) converted from RNA. The nucleic acid can be genomic DNA (gDNA). The nucleic acid can be coding DNA. The nucleic acid can be non-coding DNA. The nucleic acid can contain a target sequence that is of interest. The non-target nucleic acid can be any type of nucleic acid. The non-target nucleic acid may be the same type of nucleic acid as the target nucleic acid, but devoid of the target sequence.

In some embodiments, the target sequence is a highly diversified or mutated nucleic acid sequence. For example, the target sequence can be from a T-cell receptor, a B-cell receptor, or an NK cell receptor. In some embodiments, the target sequence can comprise a gene fusion. For example, in some embodiments, the target sequence can be a contiguous sequence of nucleic acids formed by the joining of two previously independent genes. In some embodiments, the target sequence comprises a sequence that can be difficult to access by primers. For example, in some embodiments, the target sequence comprises a sequence that contains one or more repeats, a secondary structure, a sequence that is high in GC or AT sequences, or the target sequence can be a gene with a pseudogene or multiple alleles across the genome that also bind to the primers. In some embodiments, target and non-target nucleic acids of a library used in methods of the disclosure can contain additional sequences to identify single-cell or single-molecule origin, for example, a nucleotide barcode or UMI at 3′ end of a transcript.

The method includes pre-circularization amplification. Pre-circularization amplification, as described herein, can involve amplifying target and non-target nucleic acids with primers that contain a 5′ sequence and a 3′ sequence. In some embodiments, the primers contain one or more cleavage moieties which allow for subsequent circularization. In some embodiments, the 5′ sequences contain the one or more cleave moieties. In some embodiments, the cleavage moieties comprise a uracil base. In some embodiments, sequences upstream of the one or more uracil bases of one primer is a sequence that is a reverse-complement to that of another primer used to amplify the target or non-target nucleic acid. In some embodiments, the first base of the first set of primers (Primer-1 and Primer-2) is an adenine. In some embodiments, the 3′ sequence of the first set of primers binds to templates. The binding can be either through common artificial sequences located on either side of a nucleic acids, or can be random. For example, in some embodiments, the first set of primers bind to exogenous sequences (i.e., sequence that do not occur naturally in the organism from which the target sequence is derived). In some embodiments, the primers comprise one or more modifications. In some embodiments, the 3′ ends of first set of primers can be protected from exonuclease digestion. For example, in some embodiments, the first set of primers comprise one or more modifications, wherein the one or more modifications is a phosphorothioate bond.

Pre-circularization amplification can be accomplished by binding the first set of primers (Primer-1 and Primer-2) to target and non-target nucleic acids and then extending the first set of primers using a polymerase, e.g., a DNA polymerase. In some embodiments, the DNA polymerase is a polymerase that can tolerate one or more uracil residues present in the primers. That is, in some embodiments, the DNA polymerase can extend a 3′ end of a primer despite the primer containing one or more uracil residues. Tolerance also means that polymerase can incorporate an adenine base to the newly synthesized strand that base-paired with the deoxy-uracil base on the template strand. Accordingly, pre-circularization amplification can amplify target and non-target nucleic acids while simultaneously preparing the target and non-target nucleic acids for circularization.

One surprising insight of the disclosure is that pre-circularization amplification of both target and non-target nucleic acids improves the detectability of target sequences present in the target nucleic acids. Accordingly, in some embodiments, both target and non-target nucleic acids are amplified and subsequently circularized. The presence of non-target nucleic acids in the library improves methods of identifying target sequences because the presence of the non-target nucleic acids in the library improves data quality of the sequencing libraries.

In some embodiments, pre-circularization amplification comprises a plurality of cycles of primer extension. For example, and without limiting the scope of the disclosure, in some embodiments, the plurality of cycles is between 2-100. In some embodiments, the plurality of cycles is between 2-10. In some embodiments, the plurality of cycles involves 8 cycles of primer extension. In some embodiments, pre-circularization amplification is carried out by executing a program on an automated PCR instrument. In some embodiments, the PCR program comprises: 98 degrees Celsius 5 min; 8×(98 degrees Celsius 30 seconds; 60 degrees Celsius 1 min; 72 degrees Celsius 1.5 min); hold on ambient or 4 degrees Celsius.

After pre-circularization amplification, amplicons comprising target and non-target nucleic acids are circularized. In some embodiments, the amplificons are treated with a nuclease digestion that removes the one or more uracil residues and upstream sequences from the primer strands, thus creating two sticky ends that reverse-complement to each other. The digestion can be catalyzed by an enzyme mixture. For example, the enzyme mixture can contain uracil DNA glycosylase and Endonuclease VIII, which is commercially available as USER Enzyme from New England Biolabs. After digestion, the amplicons can be subjected to a DNA ligation reaction causing the two sticky ends of each amplicon molecule to ligate, thereby forming a circularized amplicon. In some embodiments, the nuclease digestion reaction and ligation reaction are consecutive. In some embodiments, the nuclease digestion reaction and ligation reaction are concurrent.

After circularizing target and non-target nucleic acids, the library of circularized amplicons is subjected to an enrichment reaction (e.g., Enrichment-1 or Enrichment-2) to enrich for target sequences. Advantageously, as a result of circularization, known sequences (e.g., adjacent regions) are located on both sides of the target sequences. Accordingly, the known sequences of the adjacent regions can be used to selectively amplify the target sequences by extending one or more primers from the adjacent region across the target sequences thereby enriching for the target sequences. E.g., see FIGS. 1A-1, Primer-3 and Primer-4.

In some embodiments, a second set of primers (Primer-3 and Primer-4), i.e., a set of primers that are second the primers used in pre-circularization amplification (Primer-1 and Primer-2) bind to a region adjacent to the target of interest. The adjacent region can contain previously known sequences, for example a gene encoding the constant domain of the T-cell receptors, or one partner gene of a gene fusion. In some embodiments, the primers are opposite to and upstream of each other so that the amplification product encompasses the ligated region and the target sequence. In some embodiments, a single primer is used and the target sequence is enriched by a single-stranded extension reaction instead of PCR.

In some embodiments, a second enrichment reaction is performed after the first enrichment reaction. In some embodiments, the first or the second enrichment reaction is omitted. In some embodiments, the second round of enrichment is performed using a reaction mixture that contains the product from the first round of target enrichment as template and a third pair of primers (Primer-5 and Primer-6). In some embodiments, where a first round of target enrichment is not performed, the ligation reaction product is used as template with the Primer-5 and Primer-6.

In some embodiments, the third pair of primers comprises a 5′ sequence and 3′ sequence. In some embodiments, the 3′ sequence binds to the first round PCR product, and are opposite to each other, so that the amplification product encompasses the junction sequence formed by ligation and the target of interest. In some embodiments, the 3′ sequences of the third pair of primers are nested with respect to the second set of primers (Primer-3 and Primer-4) of the first round of target enrichment PCR.

In some embodiments, the 3′ sequences of the third pair of primers is identical to the second pair of primers (Primer-3 and Primer-4). In some embodiments, the 3′ sequence of one primer is non-specific to the adjacent region to the target of interest. For example, in some embodiments, the primers include random or substantially random sequences.

In some embodiments, the primers comprise index sequences. In some embodiments, a linker sequence is disclosed between a 5′ end and a 3′ end of the primer sequences, thus creating a set of staggered primers. For example, the linker sequence can include 1, 2, 3, 4, 5, 6, 7, or more nucleotides. The linker sequence can improve the quality of sequencing data by increasing diversity of nucleotides detected during the sequencing reaction.

In some embodiments, methods of the disclosure involve an indexing reaction. Indexing is a method that allows multiple libraries to be pooled and sequenced together. In some embodiments, the products of a first enrichment reaction are subjected to a second round of enrichment and indexing. In some embodiments, products of a first enrichment reaction are subjected to indexing. In some embodiments, indexing involves a PCR reaction that contains the products of a first enrichment reaction as template and a third pair of primers comprising index sequences. In some embodiments, the third pair of primers (i.e., Primer-5, and Primer-6) contain 5′ sequences that are specific to sequencing platform of choice, for example, P5 and P7 sequences from Illumina. In some embodiments, the index primers contain nucleotide barcodes for sample identification. In some embodiments, a 3′ portion of the index primers bind to products of a first enrichment reaction via 5′ consensus sequences of the two primers used in the PCR reaction. In some embodiments, a second round of target enrichment and the indexing are performed by consecutive reactions. Accordingly, in some embodiments, products of a second enrichment reaction are subject to indexing. As a person skilled in the art will readily appreciate, methods of the disclosure can include any number of enrichment reactions, e.g., 1, 2, 3, 4, 5, 6, 7, 8, 9, 10, enrichment reactions. The products of any one of those enrichment reactions can be subjected to indexing. In some embodiments, a second round of target enrichment and the indexing are performed by concurrent reactions. In some embodiments, a 3′ portion of primers are used for indexing and are specific to a region of enrichment so that the enrichment libraries are constructed without using separate Primer-5 and Primer-6.

In some embodiments, the library is subjected to a library cleanup step following indexing. In some embodiments, the library is subjected directly to sequencing following indexing. In some embodiments, the library is subjected to a library cleanup step, which is followed by sequencing. In some embodiments, products of an enrichment reaction are subjected to sequencing without indexing.

Following sequencing, sequence reads are analyzed to identify target sequences. In some embodiments, methods of the disclosure use a sequencing primer having a consensus sequence to a 3′ portion of an index primer comprising, for example, a P5 sequence. As such, in some embodiments, a Read1 cycle can identify a target sequence as a sequence beginning from the adjacent region, i.e., a known sequence, for enrichment. In some embodiments, methods of the disclosure make use of a sequencing primer that has a sequence complementary to a ligation junction. Accordingly, in some embodiments, an Index1 cycle can identify a barcode sequence located downstream of a 3′ transcript to trace the read back to a cell or molecule origin. In some embodiments, methods of the disclosure make use of a flowcell-grafted P5 sequence, or a sequencing primer reverse complementary to the 3′ of an index primer that contains a P5 sequence. Accordingly, in some embodiments, an Index2 cycle can identify a sample from which the nucleic acid corresponding to the sequence read was derived when multiple sample libraries are pooled into a single sequencing reaction. In some embodiments, a sequencing primer comprises a sequence that is reverse-complementary to the ligation junction, as such, a Read2 cycle can identify a target sequence from an upstream portion, so that the full length of a long target can be identified by stitching Read1 and Read2, which can be useful when, for example, identifying a partner gene of a gene fusion. In some embodiments, a Read2 sequence can be used for position counting. For example, in some embodiments, a Read2 sequence can be used to determine whether two sequence reads of similar sequence composition originated from two distinct molecules based on where an end (e.g., a 5′ end) of the target sequence maps on a reference. Two sequences having different ends can be identified as originating from different nucleic acid molecules (e.g., molecules of RNA).

In some embodiments, methods of the disclosure are performed with nucleic acids having a target sequence that is downstream of an adjacent region. In such embodiments, a sequencing primer comprising a sequence that is complementary to a 3′ portion of an index primer (e.g., an index primer containing a P5 sequence) is used, and as such, a Read1 cycle can be used to identify an adjacent region for enrichment to evaluate enrichment efficiency. In some embodiments, a sequencing primer comprising a sequence complementary to a ligation junction is used. In such embodiments, an Index1 cycle can be used to identify a barcode sequence located downstream of a 3′ transcript to trace either the cell or molecule origin. In some embodiments, a flowcell-grafted P5 sequence can be used, or a sequencing primer that is reverse complementary to a 3′ portion of an index primer that, for example, contains a P5 sequence can be used. In such embodiments, an Index2 cycle can be used to identify a sample to which the sequence read originated in instances where multiple sample libraries are pooled in a sequencing reaction. In some embodiments, a sequencing primer comprises a sequence that is complementary to a 3′ portion of an index primer that, for example, contains a P7 sequence is used. As such, in some embodiments, a Read2 cycle can be used to identify a target of interest from an adjacent region for enrichment.

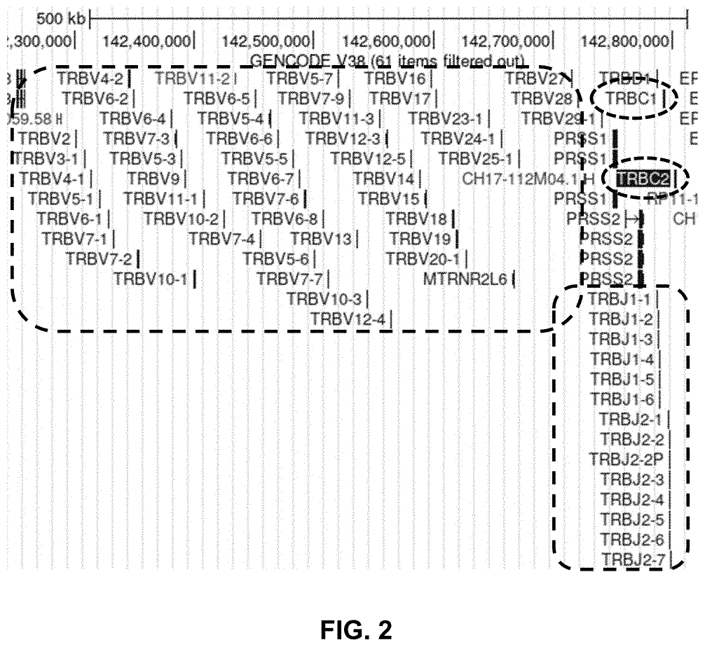

FIG. 2 shows a genomic region of the TCR beta gene to illustrates the diversity of the TCR repertoire. In particular, shown are two genes, TRBC1 and TRBC2 (dashed ovals), encoding the T cell receptor constant domain, and multiple V- and J-sequences (TRBVs and TRBJs respectively, dashed box).

T cell receptor and B cell receptor gene transcripts are often used for the purposes of TCR discovery or antibody discovery for use in cellular immunotherapy. Such methods require an efficient approach for acquiring sequence information from the variable region of TCRs and BCRs. Unfortunately, random sequencing of a standard 3′-barcoded library can be a highly inefficient means of acquiring the desired data, and if the sequence is in the 5′-end of the transcript, as in the case of the variable region of TCRs and BCRs, the desired sequences may not be extracted. Random sequencing can also suffer from trade-offs in specificity and speed when targeting exact sequences in a transcript. One major application of methods described herein is the ability for unbiased detection of different genes from complex nucleic acids. For example, when applied to a genomic region of the TCR gene, methods of this disclosure can be used to acquire accurate sequence information of a sequence region that is in diversity.

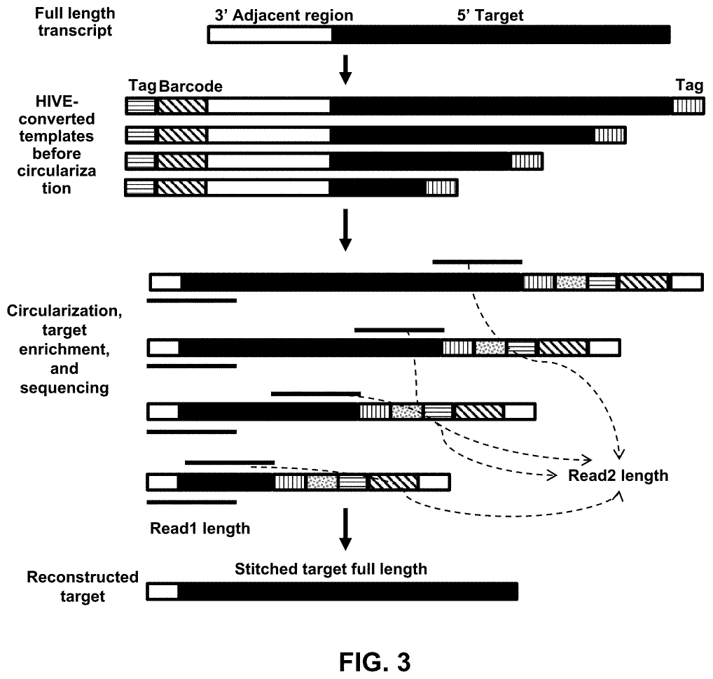

FIG. 3 illustrates a method of preparing a nucleic acid library from nucleic acids having difficult to access target regions. In some embodiments, the method can makes use of a library of full length gene transcripts. In some embodiments, the method involves converting the gene transcripts to cDNA, e.g., with reverse transcriptase. In some embodiments, the library can contain a plurality of nucleic acids, including target and non-target nucleic acids. In some embodiments, a population of target nucleic acids comprise different 5′ ends, i.e., terminate at a different nucleotide position. In some embodiments, methods of the disclosure make use of the differences in 5′ ends to count unique sequence reads and/or trace sequence reads back to an original molecule of nucleic acid. In some embodiments, methods include converting a plurality of target and non-target nucleic acids into molecules of cDNA. The method can further involve enriching for the target sequences from a portion of the circularized amplicons and subsequently, sequencing the enriched amplicons. Making reference to FIG. 3, the illustration shows a population of target nucleic acids with different 5′ ends which are detected using a pair of primers. The locations of the 5′ ends mapped to a reference and used to determine a sequence of a full target. Accordingly, sequence reads, based on their end positions, can subsequently be stitched together to determine the sequence of the full length target. In some embodiments, the stitched reads may be used for long BCR sequencing.

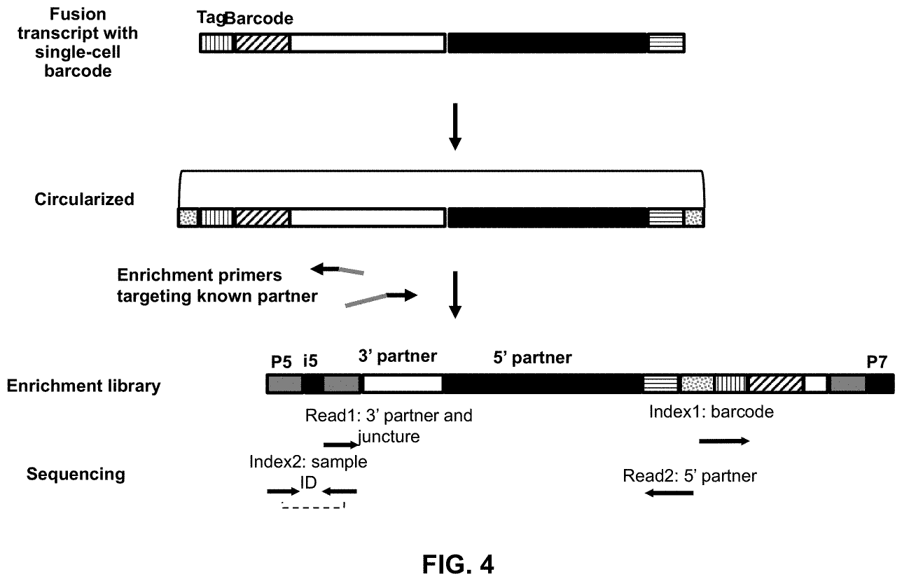

FIG. 4 illustrates a method of nucleic acid library preparation for identifying a gene fusion. A gene fusion, sometimes referenced as a fusion gene, is a hybrid gene formed from two previously independent genes. A gene fusion can be formed as a result of chromosome instability, which is hallmark of cancer. Examples of chromosome instability events that can give rise to a gene fusion include translocations, interstitial deletions, or chromosome inversions.

Methods of the disclosure are particularly useful for identifying gene fusions since methods described herein can be used to determine an unknown sequence (e.g., a sequence of a gene) based on sequence information from an adjacent sequence (e.g., sequence from one of the gene partners), as well as enrich for nucleic acids sequences that may otherwise be rare in a population of total nucleic acids. In some embodiments, the target sequence (i.e., the unknown partner of the gene fusion) comprises less than 0.1, 0.01, 0.001, 0.0001 percent of total nucleic acid present in a sample.

The method can involve pre-circularization amplification in which a plurality of target and non-target nucleic acids are amplified. The target and non-target amplicons can then be circularized. After circularization, a target sequence (e.g., the sequence of an unknown partner gene) can be determined by binding primers to the adjacent region and extending the primers across the target sequence. The enriched sequences can then be sequenced. Any sequence reads comprising sequences than can be mapped to two genes that do not occur together in a matched normal reference can be identified as a fusion. In some embodiments, methods disclosed herein are useful to screen for a plurality of gene fusion using enrichment primers that bind to any number of known gene sequences.

In some embodiments, methods of the disclosure involve identifying one or more gene fusions, which can be used to determine a health status of a subject. For example, in some embodiments, methods of the disclosure involve identifying and quantifying gene fusions. Since a gene fusion can be an indicator of chromosome instability, which is a hallmark of cancer, identifying and quantifying the gene fusions by methods described herein can be used to inform on whether a subject has cancer, or inform on a cancer treatment. In some embodiments, methods described herein are useful to identify gene fusions generated by exon skipping, which are sometimes referred to as intragenic fusions. For example, in some instances of lung cancer, exon 14 of a growth receptor gene, MET, is skipped leading to a fusion of exon 13 and exon 15. The skipping of exon 14 may be caused by many genomic events, for example point mutations, thus is harder to detect from genomic DNA than from mRNA. Methods described herein can be used to detect such mutations from mRNA or DNA.

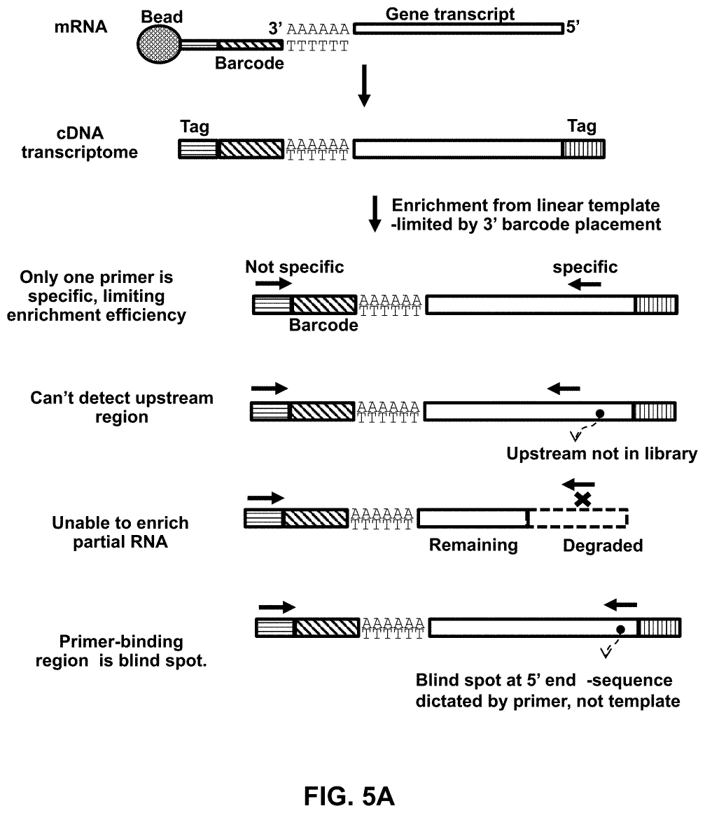

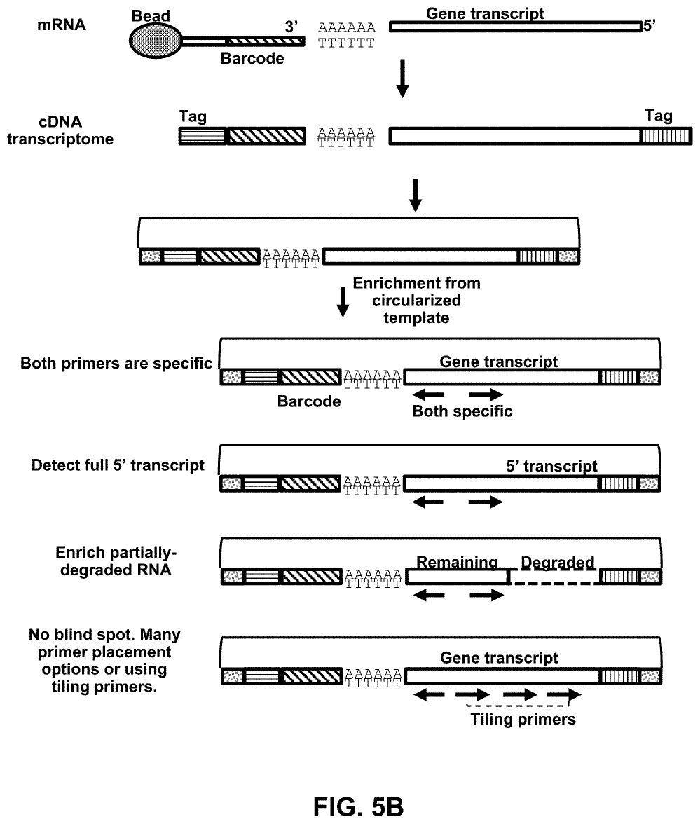

FIGS. 5A-5B illustrate certain limitations of single-cell RNA-seq that are overcome by circularization strategies disclosed herein. In particular, FIG. 5A illustrate several limitations associated with single cell RNA-seq with 3′ barcode tracing. FIG. 5B illustrates how circularization strategies of the present disclosure overcome those limitations. For example, as illustrated in FIG. 5A, conventional 3′ barcode tracing strategies generally only employ one primer that is specific for a target of interest, which limits enrichment efficiency. Conversely, by circularizing a target nucleic acid, two primers specific for a sequence (e.g., an adjacent region) can be used, thereby enhancing enrichment efficiency (see, FIG. 5B). In addition, in conventional 3′ barcode tracing strategies, sequences upstream of a primer binding site can be missed, thus sequence information can go undetected (see, FIG. 5A). Conversely, as illustrated in FIG. 5B, circularization allows a researcher or clinician to determine the sequence of the entire transcript. In addition, conventional 3′ barcode tracing strategies are generally unable to enrich or identify nucleic acids that are degraded, since degradation can inhibit primer binding. In other instances, conventional 3′ barcode tracing strategies may be unable to enrich or identify nucleic acids due to an inhibitor of a target sequence primer being present at the 5′ end of a transcript. Furthermore, conventional 3′ barcode tracing can overlook mutations present at a primer binding site because sequence information corresponding to a primer binding site is generated from the primer oligo and not the template. However, as illustrated in FIG. 5B, these limitations care overcome by circularizing transcripts and extending primers across the transcript from one or more portions of the transcripts to which primers do bind.

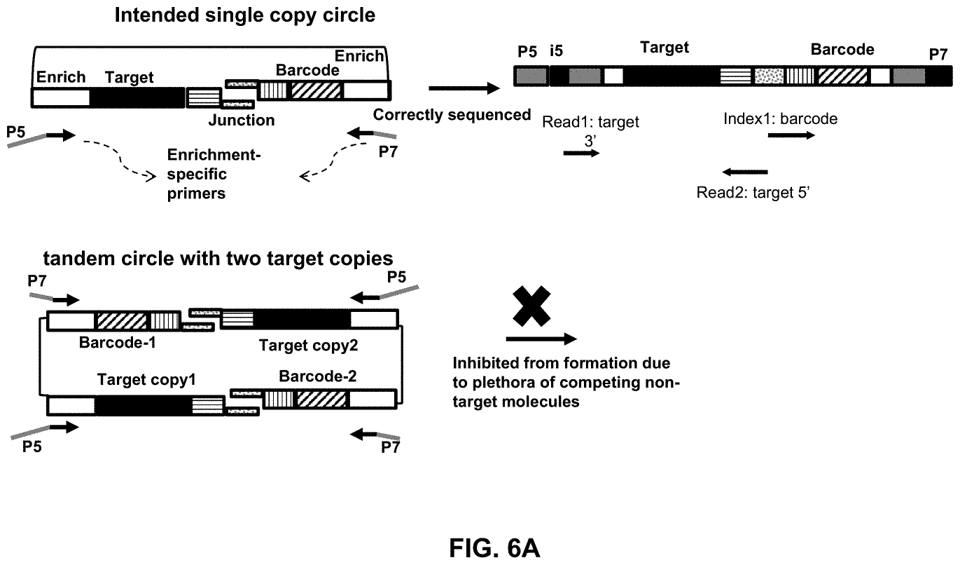

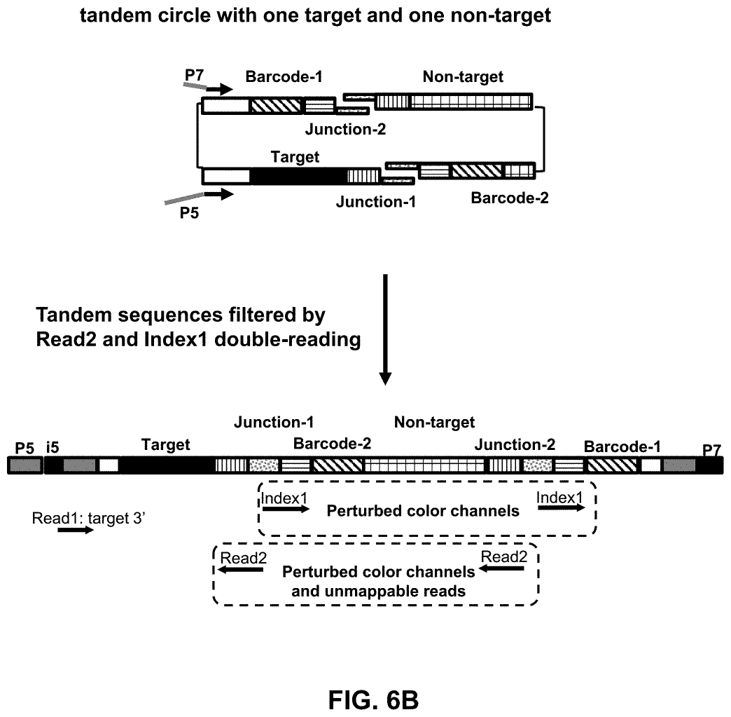

FIGS. 6A-6B diagram three scenarios for target and non-target nucleic acid circularization and the corresponding impact on sequence analysis. In a first scenario, a single nucleic acid molecule is circularized by ligation of complementary ends. The circularized molecule of the single nucleic acid can be sequenced and a single index or barcode sequence will be correctly linked to a single target of interest. In a second scenario, two distinct molecules of target nucleic acids are joined. However, as discussed above, this undesirable scenario is made rare by the plethora of competing non-target nucleic acids present in the reaction. In particular, one insight of the disclosure is to amplify non-target nucleic acids with target nucleic acids prior to circularization to reduce likelihood of two molecules of target nucleic acids joining. In some embodiments, the likelihood of two molecules of target nucleic acids joining is reduced by at least 50%, 55%, 60%, 75%, 80%, 90%, 95%, 99%, 99.9%, or more. In a third scenario, a molecule of target nucleic acid is joined with a molecule of non-target nucleic acid. However, as illustrated, any sequence reads produced from joined molecules of target and non-target nucleic acid can be filtered by Read2 and Index1 double reading. Accordingly, joined molecules of non-target and target nucleic acid have minimal to zero impact on sequence quality.

In one aspect, this disclosure provides a method of nucleic acid library preparation, the method comprising: (a) amplifying a plurality of target nucleic acids and non-target nucleic acids with first primers to generate a plurality of first amplicons, wherein: (i) each of the plurality of target nucleic acids comprises a target sequence, and an adjacent region; (ii) the first primers each comprise one or more cleavage moieties between a 5′ and 3′ end; and (iii) the amplifying comprises a plurality of cycles of primer extension with the first primers to generate a plurality of double-stranded first amplicons for each of the plurality of target and non-target nucleic acids; (b) cleaving the plurality of first amplicons at the one or more cleavage moieties to produce cleaved amplicons with self-complementary 3′ overhangs; (c) circularizing the cleaved amplicons by ligating the ends at the self-complementary 3′ overhangs to generate circularized amplicons produced from the target nucleic acids and non-target nucleic acids; and (d) for each of a plurality of circularized amplicons comprising a target sequence, amplifying at least a portion of the circularized amplicon by extending one or more second primers wherein the one or more second primers preferentially hybridize to circularized amplicons produced from the target nucleic acids; thereby producing a nucleic acid library of second amplicons enriched for the target sequences and/or complements thereof. In some embodiments, the plurality of cycles comprises between 2 and 100 cycles. For example, in some embodiments, the plurality of cycles comprises 2, 3, 4, 5, 6, 7, 8, 9, 10, 11, 12, 13, 14, 15, 16, 17, 18, 19, 20, 25, 30, 35, 40, 45, 50, or 100 cycles. In some embodiments, the plurality of cycles comprises between 5 and 10 cycles. In some embodiments, the plurality of cycles comprises 8 cycles. In some embodiments, primer binding sites for the first primers comprise exogenous sequences that are the same for each of the plurality of target and non-target nucleic acids. Accordingly, in some embodiments the first primers bind to sequences that are synthetic or do not originate from the same organism as the target sequence. In some embodiments, the exogenous primers are added to the nucleic acid in a preceding library preparation step. In some embodiments, the cleavage moiety comprises a nuclease sensitive nucleotide. In some embodiments, the cleavage moiety comprises a uracil. In some embodiments, the primers comprise a plurality of uracils. In some embodiments, cleaving the plurality of first amplicons comprises excising the uracil. In some embodiments, the first primers comprise a modification and are resistant to exonuclease digestion at one or more positions. In some embodiments, the modification comprises a phosphorothioate bond. In some embodiments, the method of nucleic library preparation involves a plurality of target nucleic acids, wherein the plurality of target nucleic acids encode at least a portion of a receptor selected from the group consisting of a T cell receptor, a B cell receptor, and a NK cell receptor. In some embodiments, the receptor is a T cell receptor or a B cell receptor, and wherein the target sequence comprises a variable region of said receptor. In some embodiments, the target nucleic acids comprise binding sites for the first primers that are located outside of the variable region. The primer binding sites can be any number of nucleotides outside the variable region. For example, in some embodiments, the primer binding sites are 1, 2, 3, 4, 5, 10, 20, 30, 40, 50, 100, 200, or more nucleotides outside the variable region. In some embodiments, amplifying the circularized amplicons comprises binding the one or more second primers to the adjacent regions. In some embodiments, (i) the one or more second primers comprise a pair of second primers that hybridize to different complementary strands in the adjacent region of one or more of the circular amplicons, and (ii) the length in the 5′ to 3′ direction along one strand of the adjacent region defined by a binding site for one primer of the pair of second primers and a complement of a binding site for the other primer of the pair of second primers is less than 5 kb apart. In some embodiments, amplifying the circularized amplicons comprises binding a single species of the one or more second primers to the adjacent regions and performing a single-stranded extension reaction with the single species of primers. In some embodiments, amplifying the circularized amplicons comprises between 2 and 30 cycles of primer extension. In some embodiments, amplifying the circularized amplicons comprises between 16 and 22 cycles of primer extension. In some embodiments, amplifying the circularized amplicons comprises 12, 13, 14, 15, 16, 17, 18, 19, 20, 21, 22, 23, 24, or 25 cycles of primer extension. In some embodiments, the method further comprises amplifying the nucleic acid library of second amplicons with pairs of third primers to produce a nucleic acid library of third amplicons. In some embodiments, wherein amplifying the nucleic acid library of second amplicons comprises between 2 and 30 cycles of primer extension. In some embodiments, amplifying the nucleic acid library of second amplicons comprises between 12 and 20 cycles of primer extension. In some embodiments, amplifying the second amplicons comprises 2, 3, 4, 5, 6, 7, 8, 9, 10, 11, 12, 13, 14, 15, 16, 17, 18, 19, 20, 21, or 22 cycles of primer extension. In some embodiments, the one of the pairs of third primers comprises a 5′ sequence and a 3′ sequence, wherein the 3′ sequence binds to a primer binding site nested with respect to the one or more second primers. In some embodiments, at least one primer of each of the pairs of third primers comprises a linker between the 5′ and 3′ sequences. In some embodiments, the pairs of third primers comprise index sequences. In some embodiments, the method further comprises sequencing the library of second amplicons, or the library of third amplicons, to generate sequence reads; and identifying one or more of the target sequences using the sequence reads. In some embodiments, identifying comprises identifying a position of a sequence corresponding to the adjacent region. In some embodiments, methods involve using position counting, wherein a 5′ end or a 3′ end of a sequence is used to count or trace a sequence back to the nucleic acid from which the sequence originates. In some embodiments, the sequencing further comprises sequencing a barcode sequence, wherein the barcode sequence identifies a sample of origin of the associated target sequence. In some embodiments, the one or more of the target sequences comprises a gene fusion. In some embodiments, the gene fusion is identified by combining a first sequence read with a second sequence read to generate a long sequence read and mapping the long sequence read to a reference genome. In some embodiments, the method further comprises measuring enrichment efficiency for the target sequence based on an analysis of sequences corresponding to the adjacent region.

In another aspect, this disclosure provides a method of gene profiling, the method comprising: (a) constructing a library comprising a plurality of double stranded molecules of target cDNA and non-target cDNA with a poly-T primer, wherein each double stranded molecule of target cDNA comprises a target sequence and an adjacent region; (b) amplifying the plurality of double stranded molecules of target cDNA and non-target cDNA with first primers that comprise cleavage moieties to generate a plurality of first amplicons; (c) cleaving the plurality of first amplicons at the cleavage moieties to produce cleaved amplicons with self-complementary 3′ overhangs; (d) circularizing the cleaved amplicons by ligating the self-complementary 3′ overhangs to generate circularized amplicons produced from the target cDNA and non-target cDNA; and (e) for each of a plurality of the circularized amplicons comprising a target sequence, amplifying a portion of the circularized amplicons by extending one or more second primers, wherein the one or more second primers preferentially hybridize to circularized amplicons produced from the target cDNA, thereby producing a nucleic acid library of second amplicons enriched for the target sequences and/or complements thereof. In some embodiments, the constructing comprises reverse transcribing mRNA from a cell with the poly-T primer and performing a template switching reaction to produce the plurality of double stranded molecules of target cDNA and non-target cDNA. In some embodiments, constructing comprises reverse transcribing mRNA from a cell with the poly-T primer and performing a random priming reaction to produce the plurality of double stranded molecules of target and non-target cDNA. The cell can be any type of cell, including a eukaryotic or prokaryotic cell. In some embodiments, the cell is an immune cell. In some embodiments, the cell is selected from the group consisting of a T-cell, a B-cell, and a NK-cell. In some embodiments, (i) the plurality of double stranded molecules of target cDNA encode at least a portion of a receptor comprising a T-cell receptor or a B-cell receptor, and (ii) the target sequence of the target cDNA comprises a variable region of said receptor. In some embodiments, amplifying duplicates the entire variable region from each of a plurality of the double stranded molecules of cDNA. In some embodiments, amplifying comprises a plurality of cycles of primer extension with the first primers. In some embodiments, the plurality of cycles comprises between 2 and 100 cycles. For example, in some embodiments, the plurality of cycles comprises 2, 3, 4, 5, 6, 7, 8, 9, 10, 11, 12, 13, 14, 15, 16, 17, 18, 19, 20, 25, 30, 35, 40, 45, 50, or 100 cycles. In some embodiments, the plurality of cycles comprises between 5 and 10 cycles. In some embodiments, the plurality of cycles comprises 8 cycles. In some embodiments, amplifying the circularized amplicons comprises between 2 and 30 cycles of primer extension. In some embodiments, amplifying the circularized amplicons comprises between 16 and 22 cycles of primer extension. In some embodiments, amplifying the circularized amplicons comprises 12, 13, 14, 15, 16, 17, 18, 19, 20, 21, 22, 23, 24, or 25 cycles of primer extension. In some embodiments, comprising amplifying the library of the second amplicons with one or more pairs of third primers to generate a library of third amplicons. In some embodiments, amplifying the library of second amplicons comprises between 12 and 15 cycles of primer extension with the one or more pairs of third primers. In some embodiments, wherein amplifying the nucleic acid library of second amplicons comprises between 10 and 30 cycles of primer extension. In some embodiments, amplifying the nucleic acid library of second amplicons comprises between 2 and 20 cycles of primer extension. In some embodiments, amplifying the second amplicons comprises 10, 11, 12, 13, 14, 15, 16, 17, 18, 19, 20, 21, or 22 cycles of primer extension. In some embodiments, the method further comprises sequencing the library of second amplicons, or the library of third amplicons, to generate sequence reads; and generating a gene profile of the cell with the sequence reads.

In another aspect, the disclosure provides a method of genotyping a T-cell or a B-cell, the method comprising: (a) amplifying one or more target nucleic acid molecules and non-target nucleic acid molecules from a T-cell or a B-cell with first primers to generate a library of first amplicons, wherein each of the one or more target nucleic acid molecules encodes a variable region of a receptor and an adjacent region (b) circularizing the first amplicons produced from the one or more target nucleic acid molecules and non-target nucleic acid molecules to generate circularized amplicons; and (c) amplifying a portion of the circularized amplicons by extending one or more primers across the variable regions to generate a nucleic acid library of second amplicons that is enriched for the variable regions. In some embodiments, amplifying comprises a plurality of cycles. In some embodiments, the plurality of cycles comprises between 2 and 100 cycles. For example, in some embodiments, the plurality of cycles comprises 2, 3, 4, 5, 6, 7, 8, 9, 10, 11, 12, 13, 14, 15, 16, 17, 18, 19, 20, 25, 30, 35, 40, 45, 50, or 100 cycles. In some embodiments, the plurality of cycles comprises between 5 and 10 cycles. In some embodiments, the plurality of cycles comprises 8 cycles. In some embodiments, amplifying the circularized amplicons comprises between 2 and 30 cycles of primer extension. In some embodiments, amplifying the circularized amplicons comprises between 16 and 22 cycles of primer extension. In some embodiments, amplifying the circularized amplicons comprises 12, 13, 14, 15, 16, 17, 18, 19, 20, 21, 22, 23, 24, or 25 cycles of primer extension. In some embodiments, each of the primers comprises a cleavage moiety. In some embodiments, circularizing the first amplicons comprises cleaving the first amplicons at the cleavage moiety to generate cleaved amplicons comprising self-complementary 3′ ends, and ligating the self-complementary 3′ ends. In some embodiments, amplifying the circularized amplicons comprises binding the one or more second primers to the adjacent regions and extending the one or more primers across the variable regions. In some embodiments, the method further comprises amplifying the library of second amplicons with one or more pairs of third primers to generate a library of third amplicons. In some embodiments, wherein amplifying the nucleic acid library of second amplicons comprises between 2 and 30 cycles of primer extension. In some embodiments, amplifying the nucleic acid library of second amplicons comprises between 12 and 20 cycles of primer extension. In some embodiments, amplifying the second amplicons comprises 10, 11, 12, 13, 14, 15, 16, 17, 18, 19, 20, 21, or 22 cycles of primer extension. In some embodiments, the method involves amplifying the nucleic acid library of second amplicons, or the library of third amplicons, to generate a sequencing library; sequencing the sequencing library to generate a plurality of sequence reads; and genotyping the T-cell or the B-cell from the plurality of sequence reads.

In another aspect, this disclosure provides a kit for performing a method of nucleic acid preparation as disclosed herein, wherein the kit comprises: a DNA polymerase that is tolerant to uracil; one or more primer pairs; and a buffer. In some embodiments, the kit further comprises at least one primer pair with sequences complementary to a portion of a T-cell receptor. In some embodiments, the kit further comprises at least one primer pair with sequences that are complementary to a portion of a house-keeping gene. In some embodiments, the kit further comprises an endonuclease and a ligase. In some embodiments, the kit further comprises sequencing primers. In some embodiments, the kit further comprises indexing primers. In some embodiments, the kit further comprises beads for performing a library cleanup reaction.

### EXAMPLES

### Example 1. Optimization of Pre-Circularization Amplification Cycles and Input Amount

Pre-circularization amplification reactions were carried out with different numbers of amplification cycles and different template concentrations to determine a PCR cycle number that saturates yield even at a low template concentration. By determining the PCR cycle number that saturates yield even at a low template concentration, the nucleic acid library preparation methods described herein are applicable to a broad range of input amounts, e.g., trace amounts of less than a picogram, data not shown. Furthermore, the methods provide for a workflow in which input does not need to be purified prior to amplification.

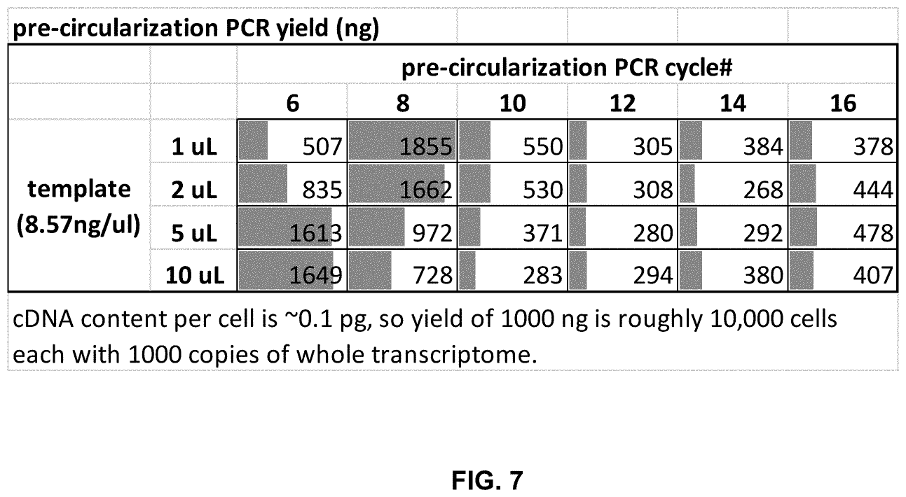

FIG. 7 shows pre-circularization amplification data collected from reactions with various PCR cycle numbers and template concentrations. Different amounts of template nucleic acids were combined with 12.5 microliters of Pre-circ Amplification Enzyme, 5 microliters of Pre-circ Primer Mix, 3.5 microliters of 10 mM Tris and PCR amplified at different cycle numbers, i.e., 6 cycles, 8 cycles, 10 cycles, 12 cycles, 14 cycles, and 16 cycles. As indicated, the amounts of template nucleic acids used in the PCR reactions was 1, 2, 5, 10 microliters of a nucleic acid product having a concentration of 8.57 nanograms/microliter. For the PCR reactions, primers for TRAC:TRBC were used at a primer ratio of 1 to 1.

The data show pre-circularization PCR reached maximum yield at 6-8 cycles for input ranging from 8 nanograms to 86 nanograms of template. The yield is equivalent to ˜1000-fold whole transcriptome saturation for 10,000 cell input.

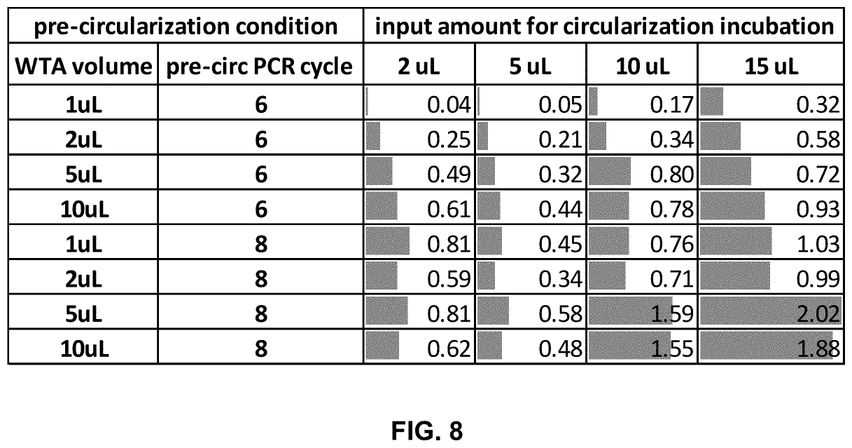

FIG. 8 shows data from experiments optimizing USER/ligate input amounts. Eight samples prepared as described above and were processed through nested PCR-1 for 21 cycles. After which, Enrichment-1 yield was evaluated. The data show increased USER/ligation input results in higher yield. Surprisingly, it was found that 8 cycles of circularization PCR was optimal even through both 6 and 8 cycles showed saturation in the assay of FIG. 7, as 8 cycles of PCR led to higher Enrichment-1 yield.

### Example 2. Optimization of Enrichment PCR Cycles to Generate a High Library Yield Enriched for Target Sequences

Additional experimentation was conducted to determine the optimal number of PCR cycles for generating a high library yield enriched for target sequences. The strategy of the experiments was to perform and saturate a first enrichment step (Enrichment-1 reaction), which has high primer concentrations, to get sufficient target over non-target nucleic acids even at the cost of some non-specific targeting, and then perform a second enrichment step (Enrichment-2 reaction) to further select and enrich for target sequences.

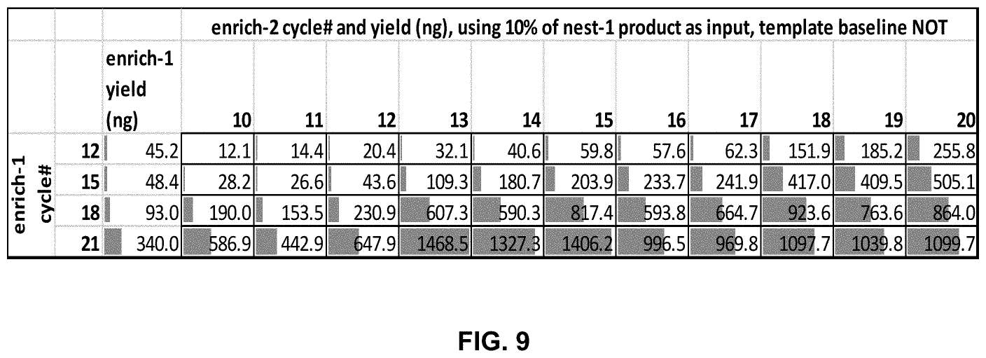

FIG. 9 shows library yields from enrichment amplification reactions carried out using different PCR cycle numbers for the first (Enrichment-1) and second enrichment step (Enrichment-2). To generate the data, a library of nucleic acids was prepared and subjected to pre-circularization PCR. For pre-circularization PCR, a reaction mixture was prepared with 4 microliters of WTA product made by using the single-cell RNAseq library preparation kit sold under the trade name Hive RNAseq (Honeycomb Biotechnologies, MA, USA), 12.5 microliters of Pre-circ Amplification Enzyme, 5 microliters of Pre-circ Primer Mix, 3.5 microliters of 10 mM Tris, for a total reaction volume of 25 microliters. The reaction mixture was then PCR amplified according to the following program: 98 degrees Celsius 5 minutes; 8 cycles of (98 degrees Celsius 30 seconds; 60 degrees Celsius 1 minute; 72 degrees Celsius 1.5 minutes) hold on ambient or 4 degrees Celsius.

Following pre-circularization PCR, the library of nucleic acids was subjected to circularization incubation. For circularization incubation, 10 microliters of the amplified nucleic acids was combined with 2 microliters of 10× Circularization Buffer, 6 microliters 10 mM Tris, and 2 microliters Circularization Enzyme for a total reaction volume of 20 microliters. The reaction mixture was incubated at 37 degrees Celsius for 30 minutes.

After circularization, a first round of enrichment (i.e., Enrichment-1 reaction) was performed. A mixture of 2 microliters of the incubation product from the circularization incubation step was combined with 5 microliters of hTCR Enrichment-1 Primer Mix, 25 microliters of PCR Enzyme, 18 microliters of 10 mM Tris, for a total reaction volume of 50 microliters. The mixture was then subjected to PCR amplification with following program: 98 degrees Celsius 5 minutes; X cycles of (98 degrees Celsius 30 seconds; 60 degrees Celsius 1 minute; 72 degrees Celsius 1.5 minutes); hold on ambient or 4 degrees Celsius, wherein X was 12, 15, 18, and 21 cycles (see FIG. 7).

For the second enrichment step (i.e., Enrichment-2/indexing PCR), a mixture of 5 microliters of the Enrichment-1 PCR product from the first enrichment step was combined with 5 microliters of hTCR Enrichment-2 Primer Mix for Illumina, 15 microliters of Unique Index for Illumina, 25 microliters of PCR Enzyme, for a total volume of 50 microliters. The mixture was then subjected to PCR amplification with following program: 98 degrees Celsius 5 minutes; X cycles of (98 degrees Celsius 30 seconds; 60 degrees Celsius 1 minute; 72 degrees Celsius 1.5 minutes); hold on ambient or 4 degrees Celsius, wherein X was 10, 11, 12, 13, 14, 15, 16, 17, 18, 19, and 20 cycles (see FIG. 9).

The data show that increasing PCR cycles during the first enrichment step (Enrichment-1) from 18 to 21 cycles substantially increases yield. Under this condition, the second enrichment step (Enrichment-2) saturates at 13 cycles

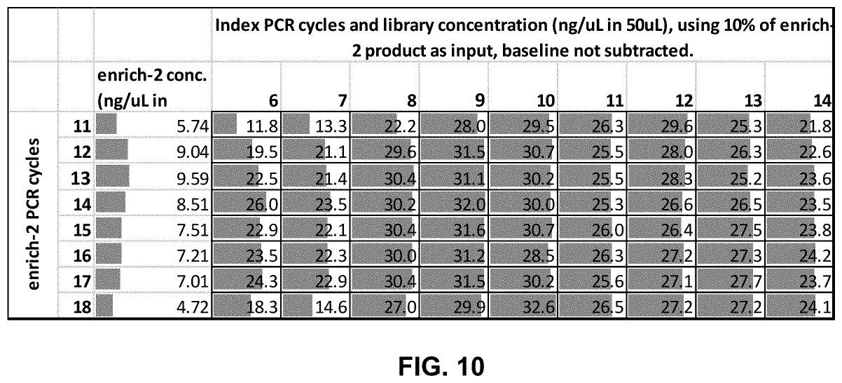

FIG. 10 shows data for identifying optimal enrichment and index cycle numbers when these two reactions are performed consecutively. In particular, the data are from experiments performed to determine optimal Enrichment-2 and index cycles using consecutive and SPRI in between. Readout is library yield. The data were collected by combining a portion of PCR-1 products prepared with 15 ul of ligation input as shown above. For the enrichment amplification, 5 ul of product was used as input for Enrichment-2, then SPRI for cleanup, and 5 uL for index PCR. The data show that Enrichment-2 yield saturates at 13 cycles and then drops, but the library yield saturates at 8-10 index PCR cycles regardless enrich-2 PCR cycles.

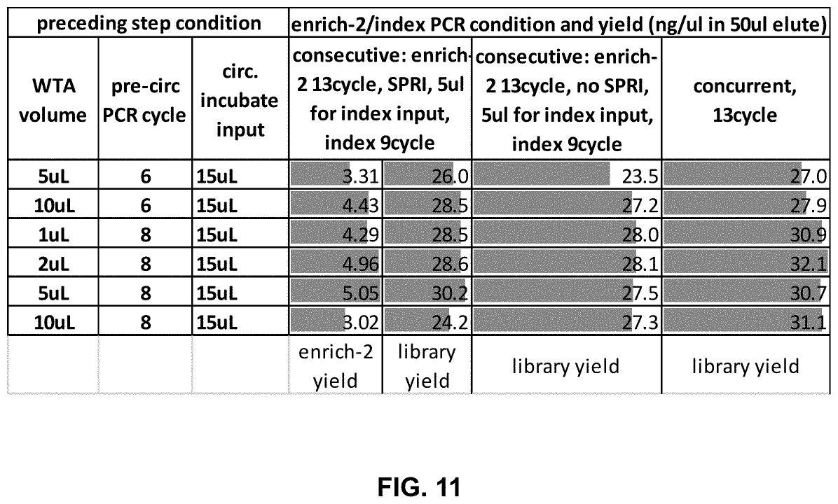

FIG. 11 shows PCR data comparing concurrent vs consecutive Enrichment-2 and Index PCR reactions. The experiment also tested whether enrich-2 PCR product needs to be SPRI-cleaned if enrich-2 and index PCR are performed consecutively. The data were collected using cell cycle numbers established above. Readout from the experiments is library yield, labchip results, and sequencing QC. The data show comparable library yields for the three conditions tested.

### Example 3. Characterization of Enrichment Libraries

To characterize enrichment libraries prepared by methods described herein, enrichment libraries were constructed using single-gene primers of two house-keeping genes (GAPDH and CHMP2A) and two TCR genes (TRAC and TRBC). A whole transcriptome RNAseq library was also constructed for comparison. The enrichment libraries and the whole transcriptome RNAseq library were then subjected to gel electrophoresis to assess the quality of target enrichment.

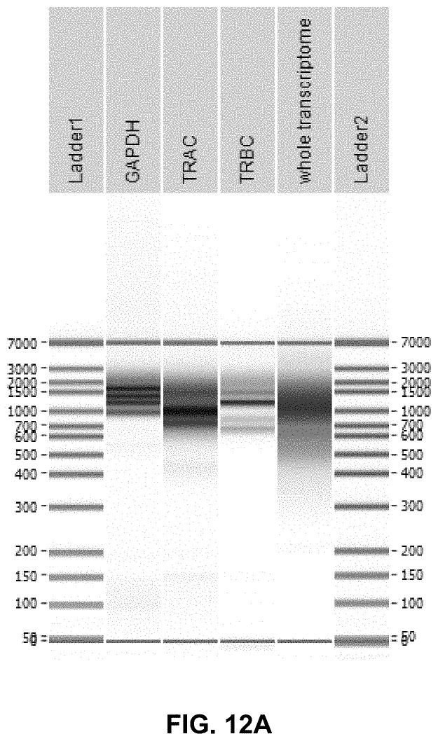

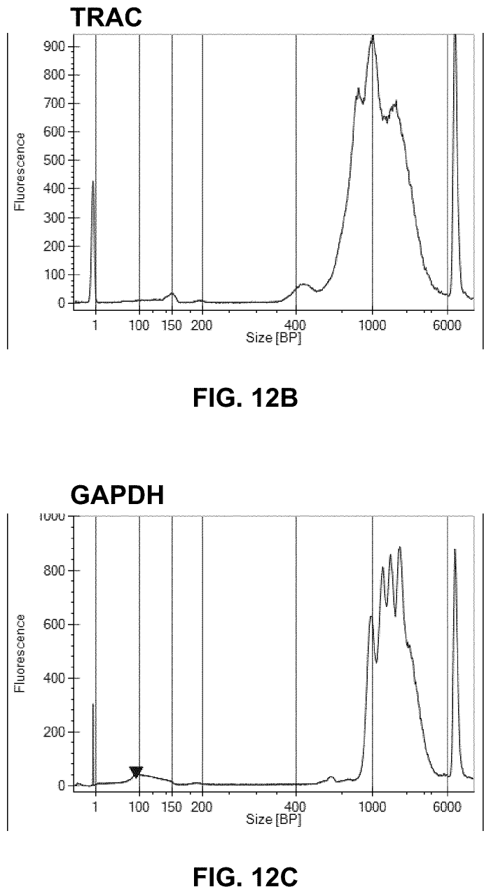

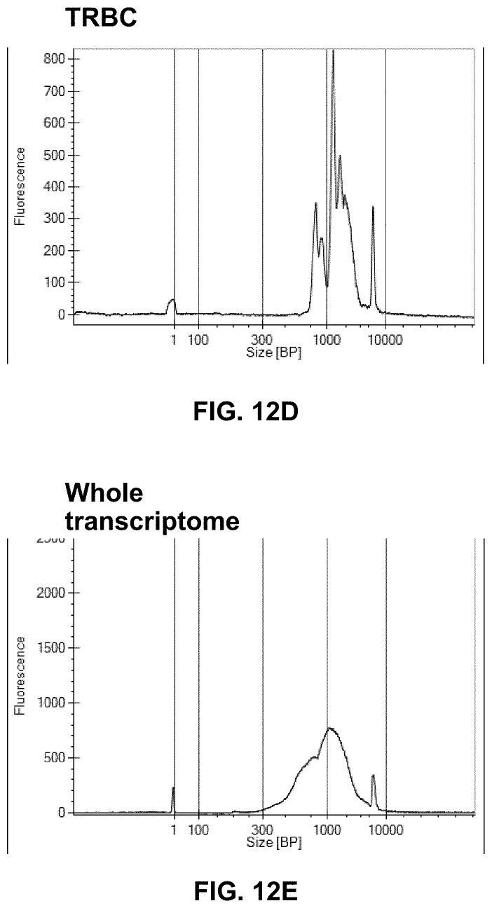

FIGS. 12A-12E show gel electrophoresis profiles of enrichment libraries prepared according to methods of the disclosure. Specifically, FIG. 12A shows a gel of multiple lanes comparing enrichment libraries enriched for GAPDH, TRAC, and TRBC, as compared to a whole transcriptome library. As shown, the enrichment libraries produce distinct bands that correspond to nucleic acid products of the enriched genes. Conversely, the whole transcriptome RNAseq library produces a smear. Individual spectral profiles of the gel for each of the libraries are shown in FIGS. 12B-E. FIG. 12B shows the gel electrophoresis profile of TRAC. FIG. 12C shows the gel electrophoresis profile of GAPDH. FIG. 12D shows the gel electrophoresis profile of TRBC. FIG. 12E shows the gel electrophoresis profile of the whole transcriptome RNAseq library. As shown, each enrichment library demonstrates a gel pattern with multiple distinctive peaks, which is indicative of enrichment. Conversely, the whole transcriptome RNAseq library fails to produce distinctive peaks.

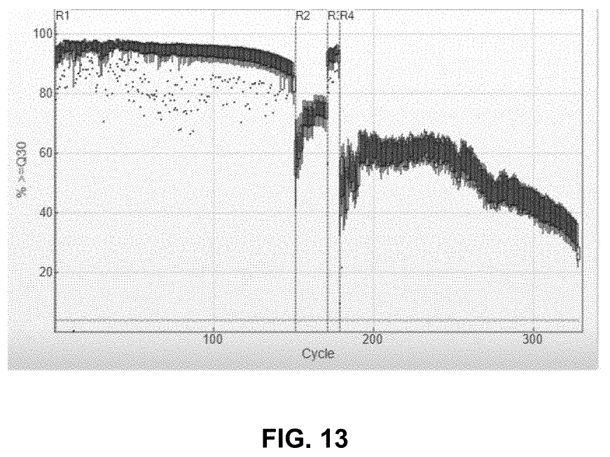

The enrichment libraries where then sequenced on an Illumina sequencing platform. Data collected from a QC analysis performed during sequencing is shown in FIG. 13.

FIG. 13 shows data of sequencing quality of enrichment libraries. The Y-axis is percent of reads that pass the Q30 (99.9% confidence) quality threshold. The X-axis indicates read cycles. ‘R1’ corresponds to Read1, ‘R2’ corresponds to Index1, ‘R3’ corresponds to Index2, and ‘R4’ corresponds to Read2. The data show that nucleic acid library preparation methods of this disclosure can successfully produce high quality sequencing libraries. In particular, the data show high quality Read1 and Index2 cycles. Index1 and Read2 quality was lower than that of Read1 and Index2, which may be attributed to perturbed double-reading (e.g., as discussed in FIGS. 6A-6B), which can be filtered.

The sequence reads were mapped to a reference and analyzed to determine reads corresponding to target sequences vs non-target sequences.

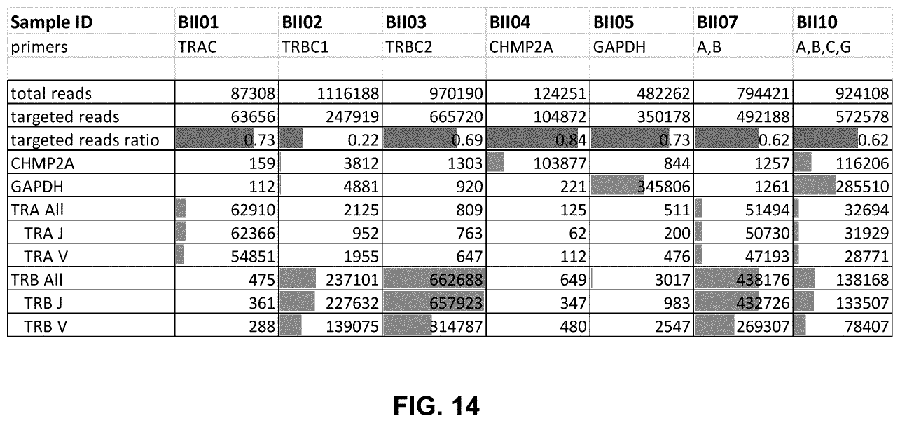

FIG. 14 shows data generated by sequencing enrichment libraries. The data show target read ratios for target nucleic acids enriched for with primers towards, i.e., genes TRAC, TRBC1, TRBC2, CHMP2A, and GAPDH. These data indicate high target read ratios (defined as reads mapped to four enriched genes over total reads), and reads mapped to individual genes that were enriched.

### Example 4. Modification of Library Prep Workflow

The data from the examples above demonstrate that when a nucleic acid library is prepared as disclosed herein with two rounds of enrichment (i.e., Enrichment-1 and Enrichment-2), the methods give rise to high quality sequencing libraries enriched for target sequences. To evaluate the impact of performing a single round of enrichment, as opposed to two rounds of enrichment, nucleic acid libraries were prepared for sequencing in which the libraries were either subjected to two rounds of enrichment (Enrichment-1 and Enrichment-2 amplification) or a single enrichment step (Enrichment-1). The libraries were then sequenced. The resultant sequence reads were mapped and target to non-target sequence reads were determined.

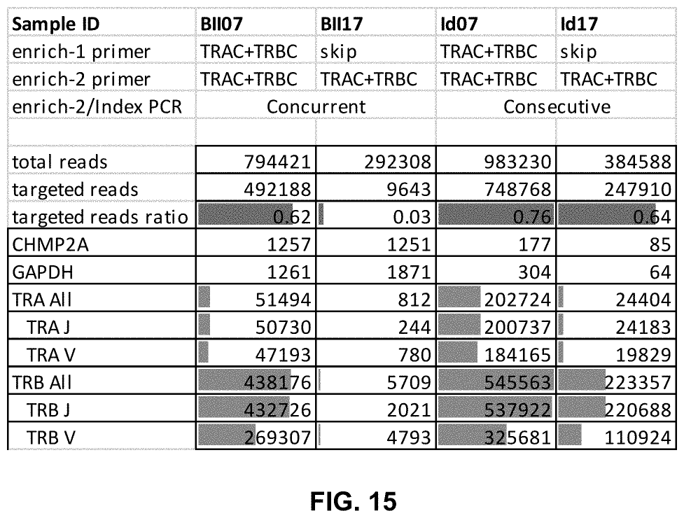

FIG. 15 shows sequence data of libraries prepared with a single enrichment reaction compared to libraries prepared with two enrichment reactions. The data show that in some instances, a single enrichment reaction can produce high quality sequencing data enriched for targets of interest.

### Example 5. Titrate TCR Alpha to Beta Primer Ratio to Adjust Read Allocations

Experiments were conducted to determine the impact of primer ratios on target vs non-target read allocations and to identify an optimal ratio of primers.

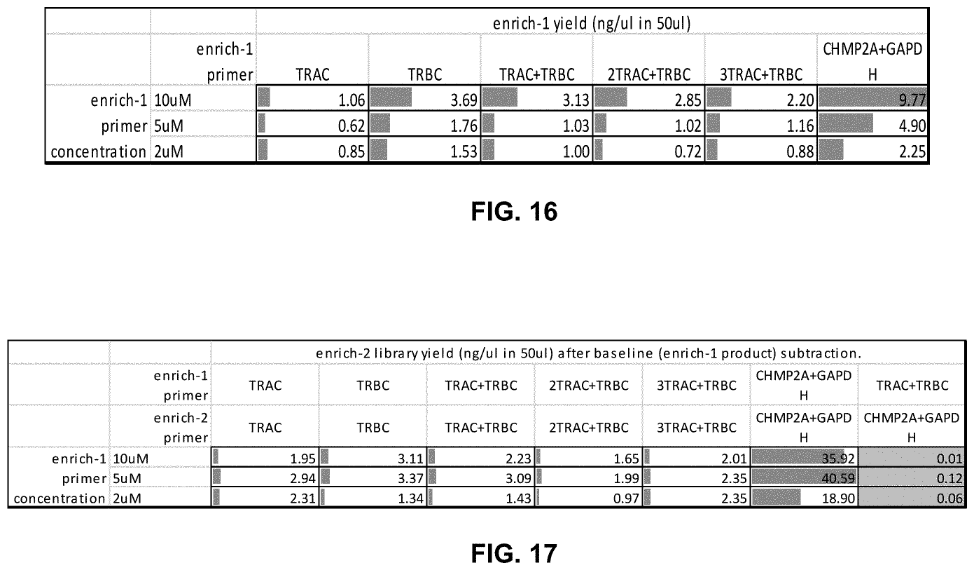

FIG. 16 shoes amplification data comparing various concentrations of primers for enrichment genes of interest. The data show high (10 uM) Enrichment-1 primer concentration is preferred, as evidence by high saturation. Different yield between genes can be due to transcript abundancy or primer efficiency.

FIG. 17 shows amplification data using different concentrations of the first set of primers (Primer-1 and Primer-2). Data show library yields are proportional to Enrichment-1 yield, confirming high Enrichment-1 primer is useful. Enrichment-1/Enrichment-2 primer cross-over has no yield after template baseline-subtraction, confirming that nested PCR is specific.

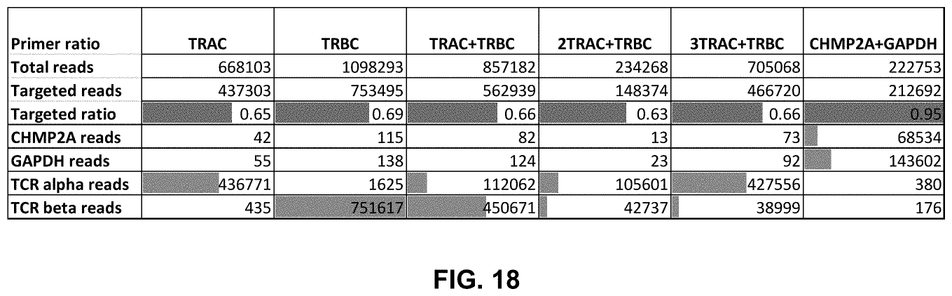

FIG. 18 shows target read ratios of sequenced libraries enriched for target sequences. In particular, FIG. 18 shows target read ratios of sequenced libraries enriched using primers for TCR alpha (TRAC), TCR beta (TRBC, with TRBC1:TRBC2 at 1:1 ratio), TCR alpha and beta at 1:1 (TRAC+TRBC), 2:1 (2TRAC+TRBC), and 3:1 (3TRAC+TRBC) ratios, and two control gene at 1:1 ratio (CHMP2A+GAPDH). Note that these ratios apply to both rounds of PCR primers, and therefore may create a compound effect. The data shows a shift of TCR alpha to beta read allocations corresponding to their primer ratio change.

### Example 6. Preparation of Nucleic Acid Libraries for Target Sequencing in Less One Day

The amount of time required to complete each step of a nucleic acid library preparation method as described herein was recorded.

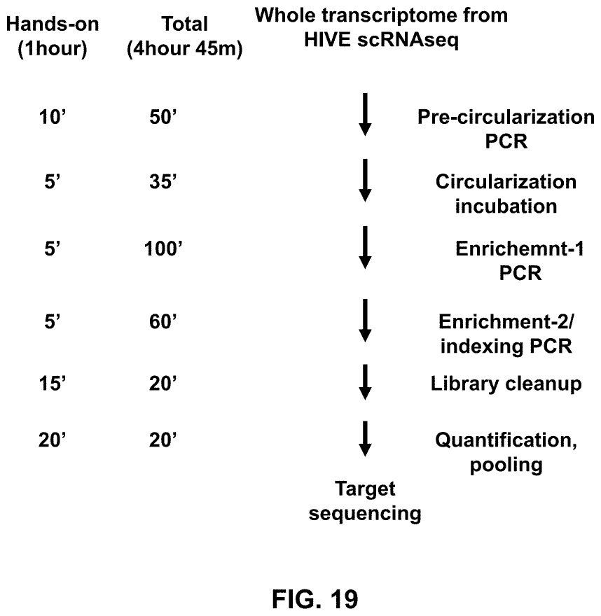

FIG. 19 shows a schematic workflow for preparing nucleic acid libraries according to aspects of this disclosure. The figure indicates the amount of time required to complete each step of the workflow, as well as hands-on time, in minutes. As shown, the amount of time in minutes to go from a library of whole transcriptome amplification products to sequencing is less than 5 hours. Accordingly, methods described herein may be useful to rapidly generate sequencing libraries enriched for targets of interest.

In particular, FIG. 19 is a schematic workflow of the protocol described below:

### Pre-Circularization PCR

1) Mix:
   4 uL WTA product from Hive RNAseq
   12.5 uL Pre-circ Amplification Enzyme
   5 uL Pre-circ Primer Mix
   3.5 uL 10 mM Tris
   25 uL total volume
2) PCR with following program:
   98 C 5 min; 8×(98 C 30s; 60 C 1 min; 72 C 1.5 min); hold on ambient or 4 C.

### Circularization Incubation

3) Mix:
   10 uL PCR product from above
   2 uL 10×Circularization Buffer
   6 uL 10 mM Tris
   2 uL Circularization Enzyme
   20 uL total volume
4) Incubate: 37 C 30 min

### Enrichment-1 PCR

5) Mix:
   2 uL incubation product from above
   5 uL hTCR Enrichment-1 Primer Mix (for the optional control reaction, replace this with human housekeeping gene control Enrichment-1 Primer Mix)
   25 uL PCR Enzyme
   18 uL 10 mM Tris
   50 uL total volume
6) PCR with following program:
   98 C 5 min; 21×(98 C 30s; 60 C 1 min; 72 C 1.5 min); hold on ambient or 4 C.

### Enrichment-2/Indexing PCR

7) Mix:
   5 uL Enrichment-1 PCR product from above
   5 uL hTCR Enrichment-2 Primer Mix for Illumina (for the optional control reaction, replace this with human housekeeping gene control Enrichment-2 Primer Mix for Illumina)
   15 uL Unique Index for Illumina
   25 uL PCR Enzyme
   50 uL total volume
8) PCR with following program:
   98 C 5 min; 13×(98 C 30s; 60 C 1 min; 72 C 1.5 min); hold on ambient or 4 C.
      Library Clean-Up with 0.8×SPRI Beads
9) Add 40 uL SPRI Beads to the indexed library, and incubate with gentle shaking for 3 minutes.
10) Place PCR plate on a magnetic stand and discard clear supernatant.
11) Wash beads twice with 80% ethanol and then air-dry for 10 minutes.
12) Elute in 50 uL 10 mM Tris.

### Sequencing

13) Product ready to be sequenced:
   a. Read 1—with standard Illumina sequencing primer, >75 cycles.
   b. Index 1—with Circ Index 1 seqP primer, 20 cycles.
   c. Index 2—with standard Illumina sequencing primer, 8 cycles.
   d. Read 2—with Circ Read 2 seqP primer, >75 cycles.
14) Use index 2 for sample demultiplexing.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

1. A method of nucleic acid library preparation, the method comprising:
(a) amplifying a plurality of target nucleic acids and non-target nucleic acids with first primers to generate a plurality of first amplicons, wherein:
(i) each of the plurality of target nucleic acids comprises a target sequence, and an adjacent region;
(ii) the first primers each comprise one or more cleavage moieties between a 5′ and 3′ end; and
(iii) the amplifying comprises a plurality of cycles of primer extension with the first primers to generate a plurality of double-stranded first amplicons for each of the plurality of target and non-target nucleic acids;
(b) cleaving the plurality of first amplicons at the one or more cleavage moieties to produce cleaved amplicons with self-complementary 3′ overhangs;
(c) circularizing the cleaved amplicons by ligating the ends at the self-complementary 3′ overhangs to generate circularized amplicons produced from the target nucleic acids and non-target nucleic acids; and
(d) for each of a plurality of circularized amplicons comprising a target sequence, amplifying at least a portion of the circularized amplicon by extending one or more second primers wherein the one or more second primers preferentially hybridize to circularized amplicons produced from the target nucleic acids;
thereby producing a nucleic acid library of second amplicons enriched for the target sequences and/or complements thereof.

2. The method of claim 1, wherein the plurality of cycles comprises between 2 and 100 cycles.

3. The method of claim 2, wherein the plurality of cycles comprises between 5 and 10 cycles.

4. The method of claim 1, wherein primer binding sites for the first primers comprise exogenous sequences that are the same for each of the plurality of target and non-target nucleic acids.

5. The method of claim 1, wherein the cleavage moiety comprises a uracil.

6. The method of claim 5, wherein cleaving the plurality of first amplicons comprises excising the uracil.

7. The method of claim 1, wherein the first primers comprise a modification and are resistant to exonuclease digestion at one or more positions; optionally wherein the modification comprises a phosphorothioate bond.

8. (canceled)

9. The method of claim 1, wherein each of the plurality of target nucleic acids encode at least a portion of a receptor selected from the group consisting of a T cell receptor, a B cell receptor, and a NK cell receptor.

10. The method of claim 9, wherein the receptor is a T cell receptor or a B cell receptor, and wherein the target sequence comprises a variable region of said receptor.

11. The method of claim 10, wherein the target nucleic acids comprise binding sites for the first primers that are located outside of the variable region.

12. The method of claim 1, wherein amplifying the circularized amplicons comprises binding the one or more second primers to the adjacent regions.

13. The method of claim 12, wherein (i) the one or more second primers comprise a pair of second primers that hybridize to different complementary strands in the adjacent region of one or more of the circular amplicons, and (ii) the length in the 5′ to 3′ direction along one strand of the adjacent region defined by a binding site for one primer of the pair of second primers and a complement of a binding site for the other primer of the pair of second primers is less than 5 kb apart.

14. The method of claim 1, wherein amplifying the circularized amplicons comprises binding a single species of the one or more second primers to the adjacent regions and performing a single-stranded extension reaction with the single species of primers.

15. The method of claim 1, wherein:
(a) amplifying the circularized amplicons comprises between 2 and 22 cycles of amplification; or
(b) the method further comprises amplifying the nucleic acid library of second amplicons with pairs of third primers to produce a nucleic acid library of third amplicons.

16. (canceled)

17. The method of claim 16, wherein amplifying the nucleic acid library of second amplicons comprises between 2 and 20 cycles of amplification.

18. The method of claim 17, wherein each one of the pairs of third primers comprises a 5′ sequence and a 3′ sequence, wherein the 3′ sequence binds to a primer binding site nested with respect to the one or more second primers.

19. The method of claim 18, wherein at least one primer of each of the pairs of third primers comprises a linker between the 5′ and 3′ sequences.

20. The method of claim 16, wherein the pairs of third primers comprise index sequences.

21. The method of claim 1, further comprising:
sequencing the library of second amplicons, or the library of third amplicons, to generate sequence reads; and
identifying one or more of the target sequences.

22. The method of claim 21, wherein:
(a) the identifying comprises identifying a position of a sequence corresponding to the adjacent region; or
(b) the sequencing further comprises sequencing a barcode sequence, wherein the barcode sequence identifies a sample of origin of the associated target sequence.

23. (canceled)

24. The method of claim 21, wherein one or more of the target sequences comprises a gene fusion.

25. The method of claim 24, wherein the gene fusion is identified by combining a first sequence read with a second sequence read to generate a chimeric sequence read wherein the first sequence read and the second sequence read mapped to two different regions of a reference genome.

26. The method of 21, further comprising measuring enrichment efficiency for the target sequence based on an analysis of sequences corresponding to the adjacent region.

27. A method of gene profiling, the method comprising:
(a) constructing a library comprising a plurality of double stranded molecules of target cDNA and non-target cDNA with a poly-T primer, wherein each double stranded molecule of target cDNA comprises a target sequence and an adjacent region;
(b) amplifying the plurality of double stranded molecules of target cDNA and non-target cDNA with first primers that comprise cleavage moieties to generate a plurality of first amplicons;
(c) cleaving the plurality of first amplicons at the cleavage moieties to produce cleaved amplicons with self-complementary 3′ overhangs;
(d) circularizing the cleaved amplicons by ligating the self-complementary 3′ overhangs to generate circularized amplicons produced from the target cDNA and non-target cDNA; and
(e) for each of a plurality of the circularized amplicons comprising a target sequence, amplifying a portion of the circularized amplicons by extending one or more second primers, wherein the one or more second primers preferentially hybridize to circularized amplicons produced from the target cDNA;
thereby producing a nucleic acid library of second amplicons enriched for the target sequences and/or complements thereof.

28. The method of claim 27, wherein the constructing comprises:
(a) reverse transcribing mRNA from a cell with the poly-T primer and performing a template switching reaction to produce the plurality of double stranded molecules of target cDNA and non-target cDNA; or
(b) reverse transcribing mRNA from a cell with the poly-T primer and then performing a second-strand synthesis reaction with a random primer.

29. (canceled)

30. The method of claim 28, wherein the cell is selected from the group consisting of a T-cell, a B-cell, and a NK-cell.

31. The method of claim 27, wherein (i) the plurality of double stranded molecules of target cDNA encode at least a portion of a receptor comprising a T-cell receptor or a B-cell receptor, and (ii) the target sequence of the target cDNA comprises a variable region of said receptor.

32. The method of claim 31, wherein amplifying duplicates the entire variable region from each of a plurality of the double stranded molecules of cDNA.

33. The method of claim 27, wherein amplifying comprises a plurality of cycles of amplification with the first primers: optionally wherein the plurality of cycles comprises between 2 and 100 cycles, or between 5 and 10 cycles.

34. (canceled)

35. (canceled)

36. The method of claim 27, wherein amplifying the circularized amplicons comprises between 2 and 22 cycles of amplification with the one or more second primers.

37. The method of claim 27, further comprising amplifying the library of the second amplicons with one or more pairs of third primers to generate a library of third amplicons.

38. The method of claim 37, wherein amplifying the library of second amplicons comprises between 2 and 15 cycles of amplification with the one or more pairs of third primers.

39. (canceled)

40. The method of claim 27, further comprising:
sequencing the library of second amplicons, or the library of third amplicons, to generate sequence reads; and
generating a gene profile of the cell with the sequence reads.

41. A method of genotyping an immune cell, the method comprising:
(a) amplifying one or more target nucleic acid molecules and non-target nucleic acid molecules from an immune cell with first primers to generate a library of first amplicons, wherein each of the one or more target nucleic acid molecules encodes a variable region of a receptor and an adjacent region of the receptor of the immune cell;
(b) circularizing the first amplicons produced from the one or more target nucleic acid molecules and non-target nucleic acid molecules to generate circularized amplicons; and
(c) amplifying a portion of the circularized amplicons by extending one or more primers across the variable regions to generate a nucleic acid library of second amplicons that is enriched for the variable regions.

42. The method of claim 41, wherein amplifying comprises a plurality of cycles; optionally wherein the plurality of cycles comprises between 5 and 10 cycles.

43. (canceled)

44. The method of claim 41, wherein each of the primers comprises a cleavage moiety.

45. The method of claim 44, wherein circularizing the first amplicons comprises cleaving the first amplicons at the cleavage moiety to generate cleaved amplicons comprising self-complementary 3′ ends, and ligating the self-complementary 3′ ends.

46. The method of claim 41, wherein amplifying the circularized amplicons comprises:
(a) binding the one or more second primers to the adjacent regions and extending the one or more primers across the variable regions; or
(b) between 2 and 22 cycles of amplification.

47. (canceled)

48. The method of claim 41, further comprising amplifying the library of second amplicons with one or more pairs of third primers to generate a library of third amplicons.

49. The method of claim 48, wherein amplifying the library of second amplicons comprises between 2 and 15 cycles of amplification.

50. The method of claim 41, further comprising:
amplifying the nucleic acid library of second amplicons, or the library of third amplicons, to generate a sequencing library;
sequencing the sequencing library to generate a plurality of sequence reads; and
genotyping the immune cell from the plurality of sequence reads.

51. The method of claim 41, wherein the immune cell is a T-cell or a B-cell.

52. A kit for performing the method of claim 1, wherein the kit comprises:
a DNA polymerase that is tolerant to uracil;
one or more primer pairs; and
a buffer.

53. The kit of claim 52, wherein the kit further comprises:
(a) at least one primer pair with sequences complementary to a portion of a T-cell receptor;
(b) at least one primer pair with sequences that are complementary to a portion of a house-keeping gene;
(c) an endonuclease and a ligase;
(d) indexing primers;
(e) beads for performing a library cleanup reaction; or
(f) sequencing primers.

54.-58. (canceled)
